(19) United States
(12) Patent Application Publication
(10) Pub. No.: US 2025/0205655 A1
(43) Pub. Date: Jun. 26, 2025

(54) AMPHIPHILIC POLYMERS TO BE USED IN BARRIERS AND PREPARATION THEREOF, BARRIERS WITH NANOPORES AND PREPARATION THEREOF

(71) Applicant: ILLUMINA, INC., San Diego, CA (US)

(72) Inventors: Antonio Conde-Gonzalez, Cambridge (GB); Charlotte Vacogne, Cambridge (GB); Oliver Uttley, Cambridge (GB); Istvan Kocsis, Cambridge (GB); Alexandre Richez, Cambridge (GB)

(73) Assignee: ILLUMINA, INC., San Diego, CA (US)

(21) Appl. No.: 18/846,193

(22) PCT Filed: Mar. 30, 2023
(86) PCT No.: PCT/EP2023/058393

§ 371 Date: Sep. 11, 2024

Related U.S. Application Data
(60) Provisional application No. 63/325,743, filed on Mar. 31, 2022.

(51) Int. Cl. B01D 69/14; B01D 69/02; B01D 71/80; B01D 71/82; C08G 77/28; C08G 81/00; C08G 83/00; C08L 83/08; C09D 187/00; C09D 201/00

(52) U.S. Cl. CPC B01D 69/144; B01D 69/02; B01D 71/80; B01D 71/82; C08G 77/28; C08G 81/00; C08G 83/004; C08L 83/08; C09D 187/005; C09D 201/005; B01D 2325/39

(57) ABSTRACT

Nanopore devices including barriers using amphiphilic units, and methods of making the same, are provided herein. In some examples, a barrier between first and second fluids includes a first layer comprising a first plurality of amphiphilic units, a second layer comprising a second plurality of the amphiphilic units and contacting the first plurality of amphiphilic units. The amphiphilic units may be substantially the same size as one another. The amphiphilic units respectively may include hydrophobic blocks and hydrophilic blocks coupled to the hydrophobic blocks.

### CROSS-REFERENCE TO RELATED APPLICATIONS

This application claims the benefit of U.S. Provisional Patent Application No. 63/325,743, filed Mar. 31, 2022 and entitled “NANOPORE DEVICES INCLUDING BARRIERS USING AMPHIPHILIC UNITS, AND METHODS OF MAKING THE SAME”, the entire contents of which are incorporated by reference herein.

### FIELD

This application relates to barriers between first and second fluids.

### BACKGROUND

A significant amount of academic and corporate time and energy has been invested into using nanopores to sequence polynucleotides. For example, the dwell time has been measured for complexes of DNA with the Klenow fragment (KF) of DNA polymerase I atop a nanopore in an applied electric field. Or, for example, a current or flux-measuring sensor has been used in experiments involving DNA captured in an α-hemolysin nanopore. Or, for example, KF-DNA complexes have been distinguished on the basis of their properties when captured in an electric field atop an α-hemolysin nanopore. In still another example, polynucleotide sequencing is performed using a single polymerase enzyme complex including a polymerase enzyme and a template nucleic acid attached proximal to a nanopore, and nucleotide analogs in solution. The nucleotide analogs include charge blockade labels that are attached to the polyphosphate portion of the nucleotide analog such that the charge blockade labels are cleaved when the nucleotide analog is incorporated into a polynucleotide that is being synthesized. The charge blockade label is detected by the nanopore to determine the presence and identity of the incorporated nucleotide and thereby determine the sequence of a template polynucleotide. In still other examples, constructs include a transmembrane protein pore subunit and a nucleic acid handling enzyme.

However, such previously known devices, systems, and methods may not necessarily be sufficiently robust, reproducible, or sensitive and may not have sufficiently high throughput for practical implementation, e.g., demanding commercial applications such as genome sequencing in clinical and other settings that demand cost effective and highly accurate operation. Accordingly, what is needed are improved devices, systems, and methods for sequencing polynucleotides, which may include using membranes having nanopores disposed therein.

### SUMMARY

Nanopore devices including barriers using amphiphilic units, and methods of making the same, are provided herein.

Some examples herein provide a barrier between first and second fluids. The barrier may include at least one layer including a plurality of amphiphilic units. The amphiphilic units may be substantially the same size as one another. The amphiphilic units respectively may include hydrophobic blocks and hydrophilic blocks coupled to the hydrophobic blocks.

In some examples, the at least one layer includes a first plurality of the amphiphilic units; and a second layer including a second plurality of the amphiphilic units and contacting the first plurality of amphiphilic units. In other examples, the at least one layer includes substantially a monolayer of the plurality of amphiphilic units.

In some examples, a molecular weight of one hydrophobic block plus a molecular weight of one hydrophilic block is between about 0.5 kDa and about 10 kDa. In some examples, each of the amphiphilic units includes from about 2 to about 10 of the hydrophobic blocks. In some examples, each of the amphiphilic units includes from about 2 to about 10 of the hydrophilic blocks.

In some examples, each of the amphiphilic units includes about the same number of hydrophilic blocks as hydrophobic blocks.

In some examples, within each of the amphiphilic units, the hydrophobic blocks are coupled to respective hydrophilic blocks at a plane. In some examples, the hydrophobic blocks and hydrophilic blocks are coupled by respective products, that are located within the plane, of a plurality of addition reactions. In some examples, the products include amide bonds. In some examples, each of the amphiphilic units includes a molecule to which the hydrophobic blocks and hydrophilic blocks are coupled.

In some examples, the hydrophilic and the hydrophobic block are connected together via an oligomer, and the structure thereby generated is called a heterograft block copolymer. In some examples, the oligomer includes an oligopeptide. In some examples, the oligopeptide has a length between about 4 and about 20 peptides. In some examples, the hydrophilic and hydrophobic block are connected together via a molecule. In some examples, the molecule includes a functionalized aliphatic molecule or a functionalized aromatic ring. In some examples, the molecule includes a functionalized benzene ring, functionalized naphthalene, functionalized anthracene, or functionalized pyrene. In some examples, the hydrophobic blocks are coupled directly to the functionalized aliphatic molecule or functionalized aromatic ring. In some examples, the hydrophilic blocks are coupled directly to the hydrophobic blocks. In some examples, the hydrophobic blocks are coupled to the molecule via first moieties, and wherein the hydrophilic blocks are coupled to the molecule via second moieties that are different than the first moieties. In some examples, the hydrophobic blocks and hydrophilic blocks alternate along a length of the molecule.

In some examples, each of the amphiphilic units includes a dendritic block copolymer. In some examples, the dendritic block copolymer includes dendrons and a core. In some examples, the dendrons include the hydrophobic blocks and the hydrophilic blocks. In some examples, the hydrophobic blocks are on different dendrons than the hydrophilic blocks. In some examples, the hydrophobic blocks are on at least some of the same dendrons as the hydrophilic blocks. In some examples, the dendrons include about the same number of hydrophobic blocks as hydrophilic blocks. In some examples, from about 2 to 4 hydrophobic dendrons are included, and from about 2 to 4 hydrophilic dendrons are included. In some examples, the hydrophilic blocks are coupled directly to the core. In some examples, the core includes a dendritic polyamide (e.g., a polypeptide as a nonlimiting example). In some examples, the dendritic polyamide includes between about 2 and about 8 branches.

In some examples, the hydrophobic blocks include poly(dimethyl siloxane). In some examples, the hydrophilic blocks include polyethylene oxide (PEO).

Some examples herein provide a method of forming a barrier between first and second fluids. The method may include obtaining amphiphilic units that are substantially the same size as one another. The amphiphilic units respectively may include hydrophobic blocks and hydrophilic blocks coupled to the hydrophobic blocks. The method may include forming at least one layer comprising a plurality of the amphiphilic units.

Some examples herein provide a method of forming an amphiphilic unit. The method may include coupling a predetermined number of hydrophobic blocks to a predetermined number of hydrophilic blocks using a predetermined number of addition reactions.

In some examples, at least some of the hydrophobic blocks include first and second moieties; and at least some the hydrophilic blocks include third and fourth moieties. In some examples, the addition reactions include reactions between the first moieties and the fourth moieties, and reactions between the second moieties and the third moieties. In some examples, the method further includes protecting the first moieties with a first protective group; and protecting the third moieties with a second protective group that is different from the first protective group.

In some examples, the predetermined number of the addition reactions includes: (a) coupling a first moiety of a first hydrophobic block to a fourth moiety of a first hydrophilic block; (b) removing the second protective group from the third moiety of that hydrophilic block; (c) coupling the third moiety of that hydrophilic block to the second moiety of another one of the hydrophobic blocks; (d) removing the first protective group from the first moiety of that hydrophobic block; and (e) coupling the first moiety of that hydrophobic block to the fourth moiety of another one of the hydrophilic blocks. The method also may include (f) repeating operations (b)-(e) a predetermined number of times.

In some examples, the first and second protective groups are selected from the group consisting of fluorenylmethoxycarbonyl (Fmoc) and tert-butyloxycarbonyl (Boc). In some examples, the first moiety and the third moiety are of the same type as one another. In some examples, the first moiety and third moiety are both amines. In some examples, the second moiety and the fourth moiety are of the same type as one another. In some examples, the fourth moiety are both carboxyls. In some examples, the hydrophobic blocks are coupled to the hydrophilic blocks via amide bonds.

In some examples, the addition reactions include: coupling the predetermined number of hydrophobic blocks to a molecule; and coupling the predetermined number of hydrophilic blocks to the molecule. In some examples, the molecule includes a predetermined number of first moieties and a predetermined number of second moieties that are different from the first moieties. In some examples, the hydrophobic blocks include respective third moieties that react with the first moieties to couple the predetermined number of hydrophobic blocks to the molecule. In some examples, the hydrophilic blocks include respective fourth moieties that react with the second moieties to couple the predetermined number of hydrophilic blocks to the molecule.

In some examples, the molecule includes a predetermined number of first moieties. In some examples, the hydrophilic blocks include respective second moieties that react with the first moieties to couple the predetermined number of hydrophilic blocks to the molecule, and third moieties. In some examples, the hydrophobic blocks include respective fourth moieties that react with the third moieties to couple the predetermined number of hydrophobic blocks to the molecule.

In some examples, the molecule includes a predetermined number of first moieties. In some examples, the hydrophobic blocks include respective second moieties that react with the first moieties to couple the predetermined number of hydrophobic blocks to the molecule, and third moieties. In some examples, the hydrophilic blocks include respective fourth moieties that react with the third moieties to couple the predetermined number of hydrophilic blocks to the molecule.

In some examples, the molecule includes a polypeptide. In some examples, the molecule includes a functionalized aliphatic molecule or a functionalized aromatic ring. In some examples, the molecule includes a dendrimer.

In some examples, the first, second, third, and fourth moieties are selected from the group consisting of: a vinyl moiety, a thiol moiety, an azide moiety, a carboxyl moiety, cysteine moiety, a lysine moiety, a succinimide moiety, a DBCO moiety, an acyl chloride moiety, and a propargyl moiety.

Some examples herein provide an amphiphilic unit including a predetermined number of hydrophobic blocks; and a predetermined number of hydrophilic blocks. In some examples, the hydrophobic and hydrophilic blocks alternate and are coupled to one another via amide bonds.

Some examples herein provide an amphiphilic unit including a molecule; a predetermined number of hydrophobic blocks coupled to the molecule via first moieties; and a predetermined number of hydrophilic blocks coupled to the molecule via second moieties that are different than the first moieties.

In some examples, the molecule includes a polypeptide. In some examples, the molecule includes a functionalized aliphatic molecule or a functionalized aromatic ring. In some examples, the molecule includes a dendrimer.

Some examples herein provide an amphiphilic unit including dendrons including a predetermined number of hydrophobic blocks and a predetermined number of hydrophilic blocks; and a core to which the dendrons are coupled.

In some examples, the core includes a polypeptide.

Some examples herein provide an amphiphilic unit that includes an elongated structure having first and second ends and a hydrophobic polymer disposed between the first and second ends. The amphiphilic unit may include a first dendritic core coupled to the first end and to two or more hydrophobic blocks; and a second dendritic core coupled to the second end and to two or more additional hydrophobic blocks.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1 schematically illustrates a cross-sectional view of an example nanopore composition and device including a barrier using amphiphilic units.

FIGS. 2A-2B schematically illustrate plan and cross-sectional views of further details of the nanopore composition and device of FIG. 1.

FIGS. 3A-3B schematically illustrate cross-sectional views of other example barriers using amphiphilic units.

FIGS. 4A-4B schematically illustrate example operations and compositions for use in preparing amphiphilic units.

FIGS. 5A-5C schematically illustrate additional example operations and compositions for use in preparing amphiphilic units.

FIGS. 6A-6D schematically illustrate additional example operations and compositions for use in preparing amphiphilic units.

FIGS. 7A-7B schematically illustrate additional example operations and compositions for use in preparing amphiphilic units.

FIG. 8 illustrates an example flow of operations in a method of forming a barrier using amphiphilic units.

FIG. 9 schematically illustrates a cross-sectional view of an example use of the composition and device of FIG. 1.

FIG. 10 schematically illustrates a cross-sectional view of another example use of the composition and device of FIG. 1.

FIG. 11 schematically illustrates a cross-sectional view of another example use of the composition and device of FIG. 1.

FIG. 12 schematically illustrates a cross-sectional view of another example use of the composition and device of FIG. 1.

FIG. 13 illustrates plots characterizing properties of barriers made in accordance with examples herein.

FIGS. 14 and 15 illustrate NMR spectra of example amphiphilic units.

FIG. 16A illustrates a voltage waveform used to assess barrier stability.

FIG. 16B illustrates the average survival time of example barriers as a function of time, when subjected the voltage waveform illustrated in FIG. 16A.

FIG. 17 schematically illustrates a cross-sectional view of another example use of the composition and device of FIG. 1.

### DETAILED DESCRIPTION

Nanopore devices including barriers using amphiphilic units, and methods of making the same, are provided herein.

For example, nanopore sequencing may utilize a nanopore that is inserted into a barrier, and that includes an aperture through which ions and/or other molecules may flow from one side of the barrier to the other. Circuitry may be used to detect a sequence, for example of nucleotides, e.g., during sequencing-by-synthesis (SBS) in which, on a first side of the barrier, a polymerase adds the nucleotides to a growing polynucleotide in an order that is based on the sequence of a template polynucleotide to which the growing polynucleotide is hybridized. The sensitivity of the circuitry may be improved by using fluids with different compositions on respective sides of the barrier, for example to provide suitable electron transport for detection on one side of the barrier, while suitably promoting activity of the polymerase on the other side of the barrier. The difference in fluidic compositions may generate an osmotic pressure that may weaken the barrier, and thus increase the likelihood that the barrier may break or leak during normal use. However, it may be difficult to insert nanopores into barriers that are too strong.

As provided herein, barriers for use in nanopore devices may include a plurality of amphiphilic units that provide suitable stability characteristics for long-term use of the device, and that also facilitate nanopore insertion so as to increase the number of usable devices during production. As explained in greater detail below, in some examples, the present amphiphilic units may include a plurality of hydrophilic blocks coupled to a plurality of hydrophobic blocks and may be substantially the same size as one another. The size of the amphiphilic units, as well as the respective lengths of the hydrophobic and/or hydrophilic blocks therein, may be selected such that the amphiphilic units assemble into a barrier having suitable stability and usability, e.g., in nanopore sequencing. For example, the length of the hydrophobic blocks may be selected such that the hydrophobic portion of the barrier has approximately the same thickness as a hydrophobic domain of the nanopore. Additionally, or alternatively, the size of the amphiphilic units may be selected so as to provide the barrier with suitable solubility, fluidity, and viscosity characteristics to permit nanopore insertion while still providing suitable stability during use.

First, some terms used herein will be briefly explained. Then, some example barriers using amphiphilic units, methods of making the same, and devices and methods using the same, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or system, the term “comprising” means that the compound, composition, or system includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. In specific, nonlimiting examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NS5B protein. Example RNA Reverse Transcriptases. A non-limiting example list to include are reverse transcriptases derived from Avian Myelomatosis Virus (AMV), Murine Moloney Leukemia Virus (MMLV) and/or the Human Immunodeficiency Virus (HIV), telomerase reverse transcriptases such as (hTERT), SuperScript™ III, SuperScript™ IV Reverse Transcriptase, ProtoScript® II Reverse Transcriptase.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may include posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, CA).

As used herein, the term “electrode” is intended to mean a solid structure that conducts electricity. Electrodes may include any suitable electrically conductive material, such as gold, palladium, silver, or platinum, or combinations thereof. In some examples, an electrode may be disposed on a substrate. In some examples, an electrode may define a substrate.

As used herein, the term “nanopore” is intended to mean a structure that includes an aperture that permits molecules to cross therethrough from a first side of the nanopore to a second side of the nanopore, in which a portion of the aperture of a nanopore has a width of 100 nm or less, e.g., 10 nm or less, or 2 nm or less. The aperture extends through the first and second sides of the nanopore. Molecules that can cross through an aperture of a nanopore can include, for example, ions or water-soluble molecules such as amino acids or nucleotides. The nanopore can be disposed within a barrier, or can be provided through a substrate. Optionally, a portion of the aperture can be narrower than one or both of the first and second sides of the nanopore, in which case that portion of the aperture can be referred to as a “constriction.” Alternatively or additionally, the aperture of a nanopore, or the constriction of a nanopore (if present), or both, can be greater than 0.1 nm, 0.5 nm, 1 nm, 10 nm or more. A nanopore can include multiple constrictions, e.g., at least two, or three, or four, or five, or more than five constrictions. nanopores include biological nanopores, solid-state nanopores, or biological and solid-state hybrid nanopores.

Biological nanopores include, for example, polypeptide nanopores and polynucleotide nanopores. A “polypeptide nanopore” is intended to mean a nanopore that is made from one or more polypeptides. The one or more polypeptides can include a monomer, a homopolymer or a heteropolymer. Structures of polypeptide nanopores include, for example, an α-helix bundle nanopore and a β-barrel nanopore as well as all others well known in the art. Example polypeptide nanopores include aerolysin, α-hemolysin, Mycobacterium smegmatis porin A, gramicidin A, maltoporin, OmpF, OmpC, PhoE, Tsx, F-pilus, SP1, mitochondrial porin (VDAC), Tom40, outer membrane phospholipase A, CsgG, and Neisseria autotransporter lipoprotein (NaIP). Mycobacterium smegmatis porin A (MspA) is a membrane porin produced by Mycobacteria, allowing hydrophilic molecules to enter the bacterium. MspA forms a tightly interconnected octamer and transmembrane beta-barrel that resembles a goblet and includes a central constriction. For further details regarding α-hemolysin, see U.S. Pat. No. 6,015,714, the entire contents of which are incorporated by reference herein. For further details regarding SP1, see Wang et al., Chem. Commun., 49:1741-1743 (2013), the entire contents of which are incorporated by reference herein. For further details regarding MspA, see Butler et al., “Single-molecule DNA detection with an engineered MspA protein nanopore,” Proc. Natl. Acad. Sci. 105: 20647-20652 (2008) and Derrington et al., “Nanopore DNA sequencing with MspA,” Proc. Natl. Acad. Sci. USA, 107:16060-16065 (2010), the entire contents of both of which are incorporated by reference herein. Other nanopores include, for example, the MspA homolog from Norcadia farcinica, and lysenin. For further details regarding lysenin, see PCT Publication No. WO 2013/153359, the entire contents of which are incorporated by reference herein.

A “polynucleotide nanopore” is intended to mean a nanopore that is made from one or more nucleic acid polymers. A polynucleotide nanopore can include, for example, a polynucleotide origami.

A “solid-state nanopore” is intended to mean a nanopore that is made from one or more materials that are not of biological origin. A solid-state nanopore can be made of inorganic or organic materials. Solid-state nanopores include, for example, silicon nitride (SiN), silicon dioxide (SiO₂), silicon carbide (SiC), hafnium oxide (HfO₂), molybdenum disulfide (MoS₂), hexagonal boron nitride (h-BN), or graphene. A solid-state nanopore may comprise an aperture formed within a solid-state membrane, e.g., a membrane including any such material(s).

A “biological and solid-state hybrid nanopore” is intended to mean a hybrid nanopore that is made from materials of both biological and non-biological origins. Materials of biological origin are defined above and include, for example, polypeptides and polynucleotides. A biological and solid-state hybrid nanopore includes, for example, a polypeptide-solid-state hybrid nanopore and a polynucleotide-solid-state nanopore.

As used herein, a “barrier” is intended to mean a structure that normally inhibits passage of molecules from one side of the barrier to the other side of the barrier. The molecules for which passage is inhibited can include, for example, ions or water soluble molecules such as nucleotides and amino acids. However, if a nanopore is disposed within a barrier, then the aperture of the nanopore may permit passage of molecules from one side of the barrier to the other side of the barrier. As one specific example, if a nanopore is disposed within a barrier, the aperture of the nanopore may permit passage of molecules from one side of the barrier to the other side of the barrier. Barriers include membranes of biological origin, such as lipid bilayers, and non-biological barriers such as solid-state membranes or substrates.

As used herein, “of biological origin” refers to material derived from or isolated from a biological environment such as an organism or cell, or a synthetically manufactured version of a biologically available structure.

As used herein, “solid-state” refers to material that is not of biological origin.

As used herein, “synthetic” refers to a membrane material that is not of biological origin (e.g., polymeric materials, synthetic phospholipids, solid-state membranes, or combinations thereof).

As used herein, a “solution” is intended to refer to a homogeneous mixture including two or more substances. In such a mixture, a solute is a substance which is dissolved in another substance referred to as a solvent. A solution may include a single solute, or may include a plurality of solutes. An “aqueous solution” refers to a solution in which the solvent is, or includes, water.

As used herein, the term “osmotic pressure” is intended to refer to the minimum pressure which needs to be applied to a solution to prevent the inward flow of its pure solvent across a semipermeable membrane. “Osmotic pressure” also refers to the measure of the tendency of a solution to take in a pure solvent by osmosis. Potential osmotic pressure is the maximum osmotic pressure that could develop in a solution if it were separated from its pure solvent by a semipermeable membrane. The osmotic pressure of a solution is based, at least in part, on the respective concentration(s) of solute(s) within that solution.

As used herein, a “polymeric membrane” refers to a synthetic barrier that primarily is composed of a polymer that is not of biological origin. In some examples, a polymeric membrane consists essentially of a polymer that is not of biological origin. A block copolymer is an example of a polymer that is not of biological origin and that may be included in the present barriers. A hydrophobic polymer with ionic end groups is another example of a polymer that is not of biological origin and that may be included in the present barriers. Because the present barriers relate to polymers that are not of biological origin, the terms “polymeric membrane,” “membrane,” and “barrier” may be used interchangeably herein when referring to the present barriers, even though the terms “barrier” and “membrane” generally may encompass other types of materials as well.

As used herein, the term “block copolymer” is intended to refer to a polymer having at least a first portion or “block” that includes a first type of monomer, and at least a second portion or “block” that is coupled directly or indirectly to the first portion and includes a second, different type of monomer. The first portion may include a polymer of the first type of monomer, or the second portion may include a polymer of the second type of monomer, or the first portion may include a polymer of the first type of monomer and the second portion may include a polymer of the second type of monomer. The first portion optionally may include an end group with a hydrophilicity that is different than that of the first type of monomer, or the second portion optionally may include an end group with a hydrophilicity that is different than that of the second type of monomer, or the first portion optionally may include an end group with a hydrophilicity that is different than that of the first type of monomer and the second portion optionally may include an end group with a hydrophilicity that is different than that of the second type of monomer. The end groups of any hydrophilic blocks may be located at an outer surface of a barrier formed using such hydrophilic blocks. Depending on the particular configuration, the end groups of any hydrophobic blocks may be located at an inner surface of the barrier or at an outer surface of a barrier formed using such hydrophobic blocks.

Block copolymers include, but are not limited to, diblock copolymers and triblock copolymers.

A “diblock copolymer” is intended to refer to a block copolymer that includes, or consists essentially of, first and second blocks coupled directly or indirectly to one another. The first block may be hydrophilic and the second block may be hydrophobic, in which case the diblock copolymer may be referred to as an “AB” copolymer where “A” refers to the hydrophilic block and “B” refers to the hydrophobic block.

A “triblock copolymer” is intended to refer to a block copolymer that includes, or consists essentially of, first, second, and third blocks coupled directly or indirectly to one another. The first and third blocks may include, or may consist essentially of, the same type of monomer (repeating unit) as one another, and the second block may include a different type of monomer (repeating unit). In some examples, the first block may be hydrophobic, the second block may be hydrophilic, and the third block may be hydrophobic and includes the same type of monomer as the first block, in which case the triblock copolymer may be referred to as a “BAB” copolymer where “A” refers to the hydrophilic block and “B” refers to the hydrophobic blocks. In other examples, the first block may be hydrophilic, the second block may be hydrophobic, and the third block may be hydrophilic and includes the same type of monomer as the first block, in which case the triblock copolymer may be referred to as an “ABA” copolymer where “A” refers to the hydrophilic blocks and “B” refers to the hydrophobic block.

The particular arrangement of molecules of polymer chains (e.g., block copolymers) within a polymeric membrane may depend, among other things, on the respective block lengths, the type(s) of monomers used in the different blocks, the relative hydrophilicities and hydrophobicities of the blocks, the composition of the fluid(s) within which the membrane is formed, and/or the density of the polymeric chains within the membrane. During formation of the membrane, these and other factors generate forces between molecules of the polymeric chains which laterally position and reorient the molecules in such a manner as to substantially minimize the free energy of the membrane. The membrane may be considered to be substantially “stable” once the polymeric chains have completed these rearrangements, even though the molecules may retain some fluidity of movement within the membrane.

As used herein, the term “hydrophobic” is intended to mean tending to exclude water molecules. Hydrophobicity is a relative concept relating to the polarity difference of molecules relative to their environment. Non-polar (hydrophobic) molecules in a polar environment will tend to associate with one another in such a manner as to reduce contact with polar (hydrophilic) molecules to a minimum to lower the free energy of the system as a whole.

As used herein, the term “hydrophilic” is intended to mean tending to bond to water molecules. Polar (hydrophilic) molecules in a polar environment will tend to associate with one another in such a manner as to reduce contact with non-polar (hydrophobic) molecules to a minimum to lower the free energy of the system as a whole.

As used herein, the term “amphiphilic” is intended to mean having both hydrophilic and hydrophobic properties. For example, a block copolymer that includes a hydrophobic block and a hydrophilic block may be considered to be “amphiphilic.” Illustratively, AB copolymers, ABA copolymers, and BAB copolymers all may be considered to be amphiphilic. Additionally, molecules including a hydrophobic polymer coupled to ionic end groups may be considered to be amphiphilic.

As used herein, the term “amphiphilic unit” is intended to refer to a discrete element that has at least hydrophilic element and at least one hydrophobic element. For example, a block copolymer that includes a hydrophobic block and a hydrophilic block may be considered to be an “amphiphilic unit.” Amphiphilic units may include any suitable number of hydrophobic and hydrophilic elements. For example, an amphiphilic unit may include one hydrophobic unit or a plurality of hydrophobic units, and may include one hydrophilic unit or a plurality of hydrophilic units. Within an amphiphilic unit, the hydrophobic unit(s) may be coupled to the hydrophilic unit(s) in any suitable manner. For example, the hydrophobic blocks may be coupled to respective hydrophilic blocks at a plane that is substantially parallel to the layer(s) of the barrier, or both types of blocks may be provided as parts of a dendritic block copolymer.

As used herein, a “dendrimer” is intended to refer to a polymer in which the atoms are arranged in multiple branches, or “dendrons,” which extend from a central region, or “core.” A “dendritic block copolymer” refers to a dendrimer in which the branches respectively are, or include, hydrophilic and/or hydrophobic blocks. The core of a dendritic block copolymer may include a branched molecule, such as a polymer. In some examples, a core of a dendritic block copolymer may include a “dendritic polyamide” which is intended to refer to a branched molecule including amide bonds and to which hydrophobic and/or hydrophilic blocks may be coupled so as to form dendrons. A nonlimiting example of a dendritic polyamide is a “dendritic polypeptide,” which is intended to refer to a branched polypeptide to which hydrophobic and/or hydrophilic blocks may be coupled so as to form dendrons.

As used herein, an “heterograft block copolymer” is intended to refer to a polymer in which different polymer blocks (A blocks that may be hydrophilic and B block that may be hydrophobic) are connected to one another at a central backbone, from which pendant chains are formed by the A blocks and the B blocks in an alternating pattern. This central backbone may be long or short—in the latter case it will be referred to as an oligomer—and it may have different backbone chemistries (e.g., polypeptide). In some examples, the core of a heterograft block copolymer may include a molecule to which hydrophobic and/or hydrophilic blocks may be coupled. In other examples, the hydrophobic and hydrophilic blocks may be coupled to one another to form the backbone. In examples in which the heterograft block copolymer is referred to as “alternating,” it is meant that A blocks and B blocks substantially alternate with one another along the length of the backbone.

In some examples, the structure of a dendrimer may have a point symmetry. In some examples, the structure of a heterograft block copolymer may have a line symmetry.

As used herein, a “solution” is intended to refer to a homogeneous mixture including two or more substances. In such a mixture, a solute is a substance which is uniformly dissolved in another substance referred to as a solvent. A solution may include a single solute, or may include a plurality of solutes. Additionally, or alternatively, a solution may include a single solvent, or may include a plurality of solvents. An “aqueous solution” refers to a solution in which the solvent is, or includes, water.

As used herein, the term “electroporation” means the application of a voltage across a membrane such that a nanopore is inserted into the membrane.

As used herein, the term “linker” is intended to mean a moiety, molecule, or molecules via which one element is attached to another element. Linkers may be covalent, or may be non-covalent. Nonlimiting examples of covalent linkers include moieties such as alkyl chains, polyethers, amides, esters, aryl groups, polyaryls, and the like. Nonlimiting examples of noncovalent linkers include host-guest complexation, cyclodextrin/norbornene, adamantane inclusion complexation with β-CD, DNA hybridization interactions, streptavidin/biotin, and the like.

As used herein, the terms “PEO”, “PEG”, “poly(ethylene oxide)”, and “poly(ethylene glycol)” are intended to be used interchangeably and refer to a polymer that comprises—[CH₂—CH₂—O]ₙ—. In some examples, n is between about 2 and about 100.

As used herein, the term “barrier support” is intended to refer to a structure that can suspend a barrier. When the barrier is referred to as a membrane, the barrier support may be referred to as a “membrane support.” A barrier support may define an aperture, such that a first portion of the barrier is suspended across the aperture, and a second portion of the barrier is disposed on, and supported by, the barrier. The barrier support may include any suitable arrangement of elements to define an aperture and suspend the barrier across the aperture. In some examples, a barrier support may include a substrate having an aperture defined therethrough, across which aperture the barrier may be suspended. Additionally, or alternatively, the barrier support may include one or more first features (such as one or more lips or ledges of a well within a substrate) that are raised relative to one or more second features (such as a bottom surface of the well), wherein a height difference between (a) the one or more first features and (b) the one or more second features defines an aperture across which a barrier may be suspended. The aperture may have any suitable shape, such as a circle, an oval, a polygon, or an irregular shape. The barrier support may include any suitable material or combination of materials. For example, the barrier support may be of biological origin, or may be solid state. Some examples, the barrier support may include, or may consist essentially of, an organic material, e.g., a curable resin such as SU-8; polytetrafluoroethylene (PTFE), poly(methyl methacrylate) (PMMA), parylene, or the like. Additionally, or alternatively, various examples, the barrier support may include, or may consist essentially of, an inorganic material, e.g., silicon nitride, silicon oxide, or molybdenum disulfide.

As used herein, the term “annulus” is intended to refer to a liquid that is adhered to a barrier support, located within a barrier, and extends partially into an aperture defined by the barrier support. As such, it will be understood that the annulus may follow the shape of the aperture of the barrier, e.g., may have the shape of a circle, an oval, a polygon, or an irregular shape.

### Nanopore Devices Including Barriers Using Amphiphilic Units, and Methods of Making the Same

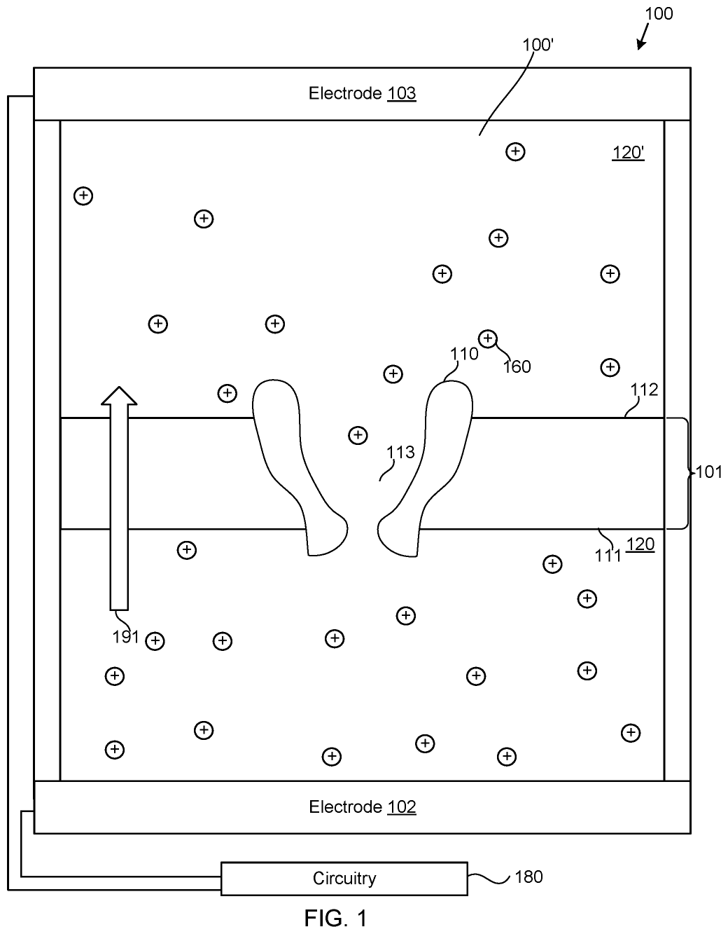

Some example devices including barriers using amphiphilic units, and methods of making the same, will be described with FIGS. 1, 2A-2B, 3A-3B, 4A-4B, 5A-5C, 6A-6D, 7A-7B, and 8.

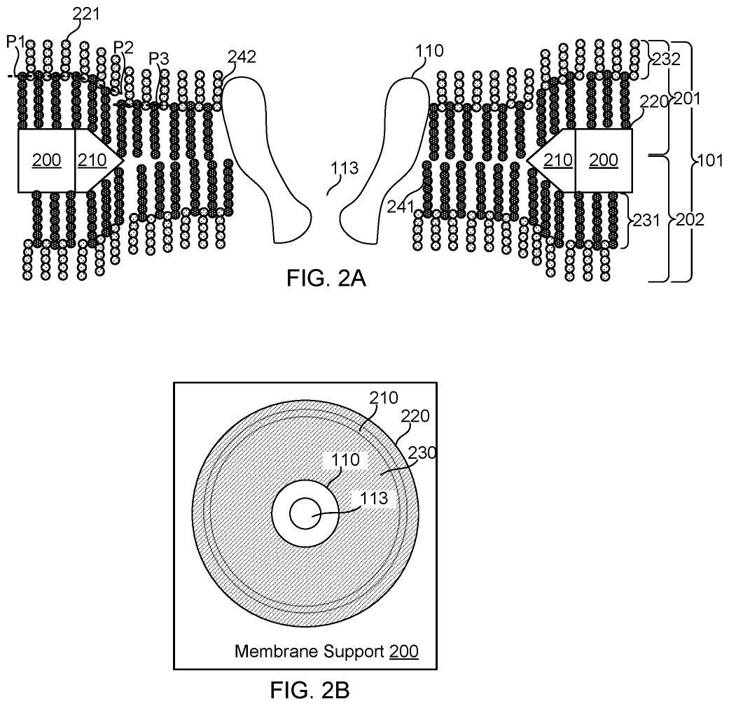

FIG. 1 schematically illustrates a cross-sectional view of an example nanopore composition and device 100 including a barrier using amphiphilic units. Device 100 includes fluidic well 100′ including polymeric membrane (barrier) 101 having first (trans) side 111 and second (cis) side 112, first fluid 120 within fluidic well 100′ and in contact with first side 111 of the membrane, and second fluid 120′ within the fluidic well and in contact with the second side 112 of the membrane. Polymeric membrane 101 may have any suitable structure that normally inhibits passage of molecules from one side of the membrane to the other side of the membrane, e.g., that normally inhibits contact between fluid 120 and fluid 120′. Illustratively, the amphiphilic units of polymeric membrane 101 may be substantially the same size as one another, respectively may include hydrophobic blocks and hydrophilic blocks coupled to the hydrophobic blocks, and may have a structure such as described in greater detail below with reference to FIGS. 2A-2B, 3A-3B, 4A-4B, 5A-5C, 6A-6D, or 7A-7B. As provided herein, the amphiphilic units may be configured so as to provide membranes having desirable stability and usability characteristics.

First fluid 120 may have a first composition including a first concentration of a salt 160, which salt may be represented for simplicity as positive ions although it will be appreciated that counterions also may be present. Second fluid 120′ may have a second composition including a second concentration of the salt 160 that may be the same as, or different, than the first concentration. Any suitable salt or salts 160 may be used in first and second fluids 120, 120′, e.g., ranging from common salts to ionic crystals, metal complexes, ionic liquids, or even water-soluble organic ions. For example, the salt may include any suitable combination of cations (such as, but not limited to, H, Li, Na, K, NH₄, Ag, Ca, Ba, and/or Mg) with any suitable combination of anions (such as, but not limited to, OH, Cl, Br, I, NO₃, ClO₄, F, SO₄, and/or CO₃²⁻ . . . ). In one nonlimiting example, the salt includes potassium chloride (KCl). It will also be appreciated that the first and second fluids optionally may include any suitable combination of other solutes. Illustratively, first and second fluids 120, 120′ may include an aqueous buffer (such as N-(2-hydroxyethyl)piperazine-N′-2-ethanesulfonic acid (HEPES), commercially available from Fisher BioReagents).

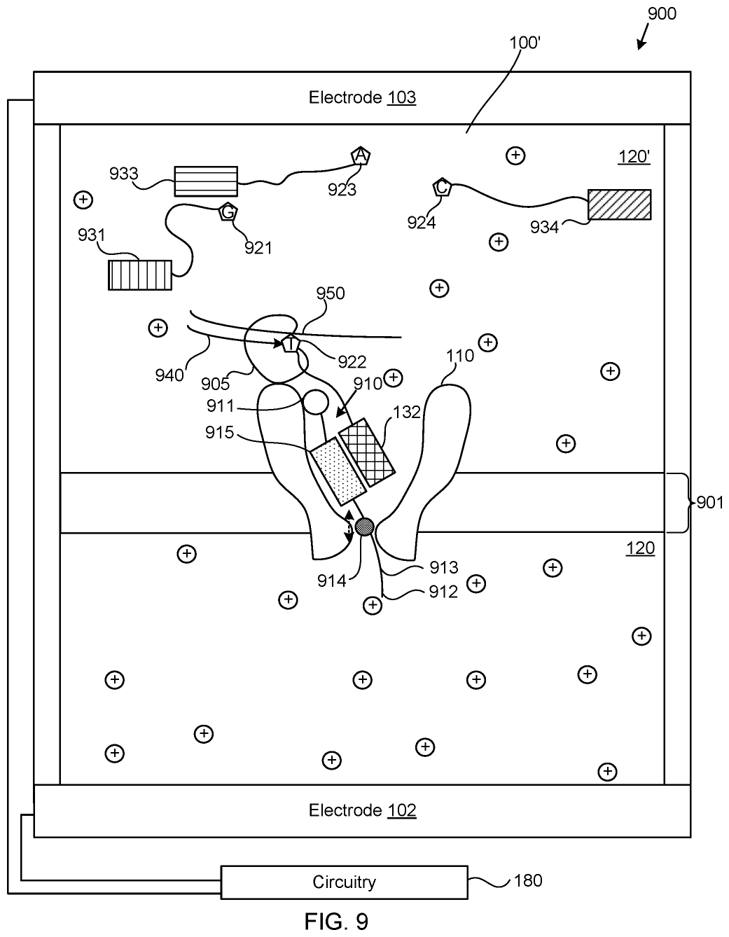

Still referring to FIG. 1, in some examples provided herein, device 100 optionally further may include nanopore disposed within barrier 101 and providing aperture 113 fluidically coupling first side 111 to second side 112. As such, aperture 113 of nanopore 110 may provide a pathway for fluid 120 and/or fluid 120′ to flow through barrier 101. For example, a portion of salt 160 may move from second side 112 of barrier 101 to first side 111 of the barrier through aperture 113. Nanopore 110 may include a solid-state nanopore, a biological nanopore (e.g., MspA such as illustrated in FIG. 1), or a biological and solid-state hybrid nanopore. Nonlimiting examples and properties of barriers and nanopores are described elsewhere herein, as well as in U.S. Pat. No. 9,708,655, the entire contents of which are incorporated by reference herein. In a manner such as illustrated in FIG. 1, device 100 optionally may include first electrode 102 in contact with first fluid 120, second electrode 103 in contact with second fluid 120′, and circuitry 180 in operable communication with the first and second electrodes and configured to detect changes in an electrical characteristic of the aperture. Such changes may, for example, be responsive to any suitable stimulus. Indeed, it will be appreciated that the present methods, compositions, and devices may be used in any suitable application or context, including any suitable method or device for sequencing, e.g., polynucleotide sequencing. As provided herein, the amphiphilic units of the barrier may provide the barrier with sufficient stability for use over a desired period of time, e.g., for use over the course of sequencing, e.g., sequencing a polynucleotide, in a manner such as described with reference to FIGS. 9-12 and 17.

For example, FIGS. 2A-2B schematically illustrate plan and cross-sectional views of further details of the nanopore composition and device of FIG. 1. As illustrated in FIG. 2A, membrane 101 may include first layer 201 including a first plurality of amphiphilic units 221 and second layer 202 including a second plurality of the amphiphilic units and contacting the first plurality of amphiphilic units. In the nonlimiting example illustrated in FIG. 2A, the amphiphilic units 221 respectively may include a plurality of hydrophobic “B” blocks 231 (within which circles 241 with darker fill represent hydrophobic monomers) and a plurality of hydrophilic “A” blocks 232 (within which circles 242 with lighter fill represent hydrophilic monomers) coupled directly or indirectly thereto. The amphiphilic units may be substantially the same size as one another, e.g., may have substantially the same molecular mass as one another. For example, the amphiphilic units 221 may include about the same number of hydrophilic A blocks 232 as one another and may include about the same number of hydrophobic B blocks 231 as one another. Illustratively, each of the amphiphilic units may include from about 2 to about 10 of the hydrophobic blocks 231. Additionally, or alternatively, each of the amphiphilic units may include from about 2 to about 10 of the hydrophilic blocks 232. The hydrophobic and hydrophilic blocks may have any suitable respective size. In one nonlimiting example, a molecular weight of one hydrophobic block 231 plus a molecular weight of one hydrophilic block 232 may be between about 0.5 kDa and about 10 kDa. It will be appreciated that these example ranges of numbers of hydrophobic and hydrophilic blocks, and example molecular weights, apply equally to other examples provided herein.

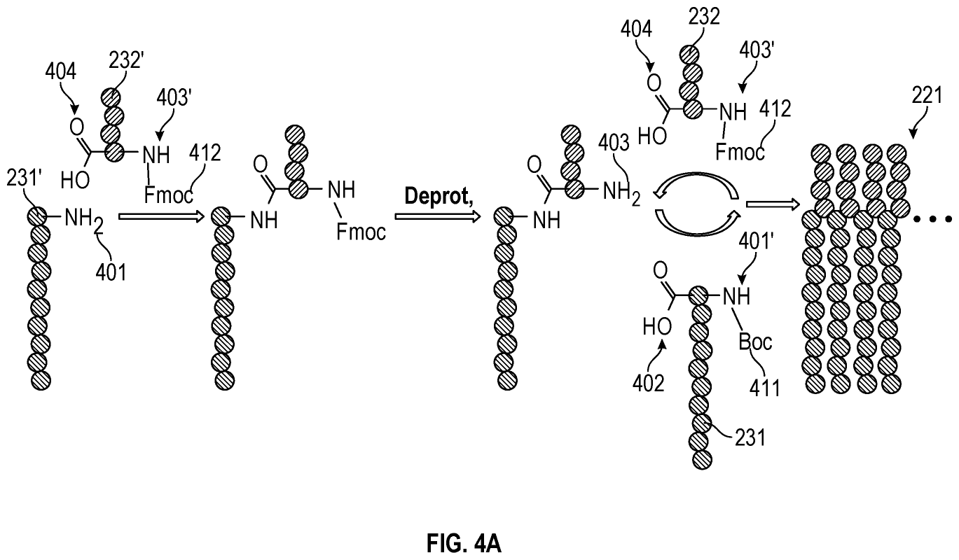

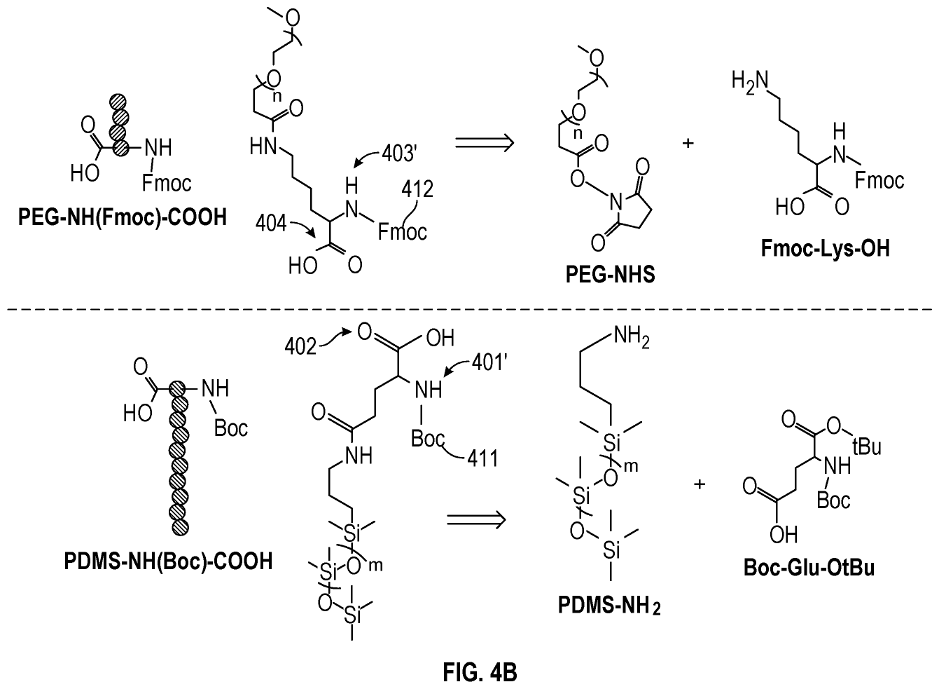

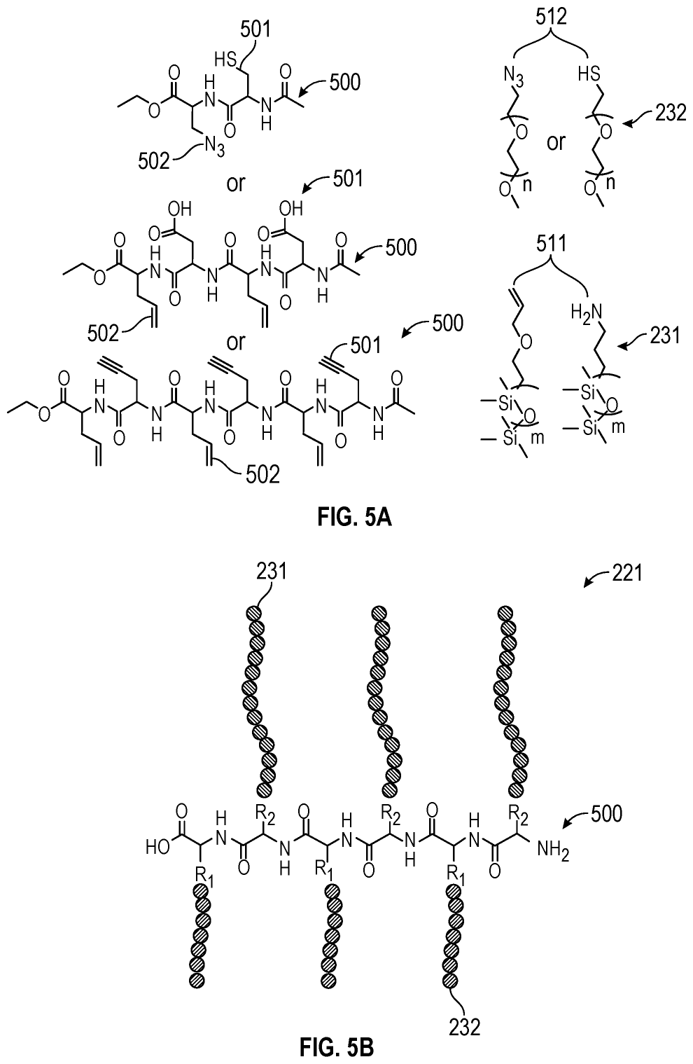

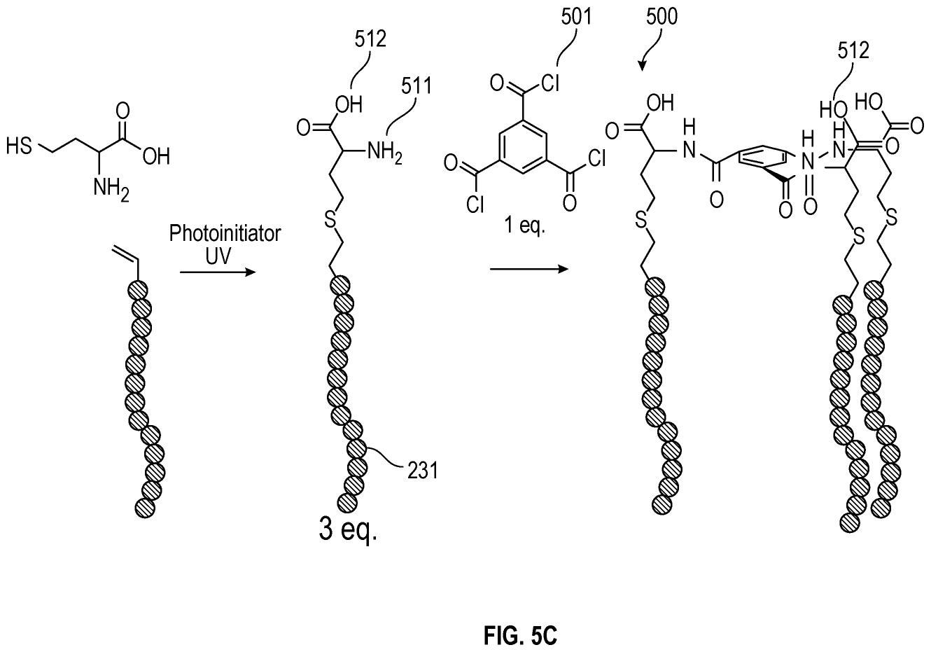

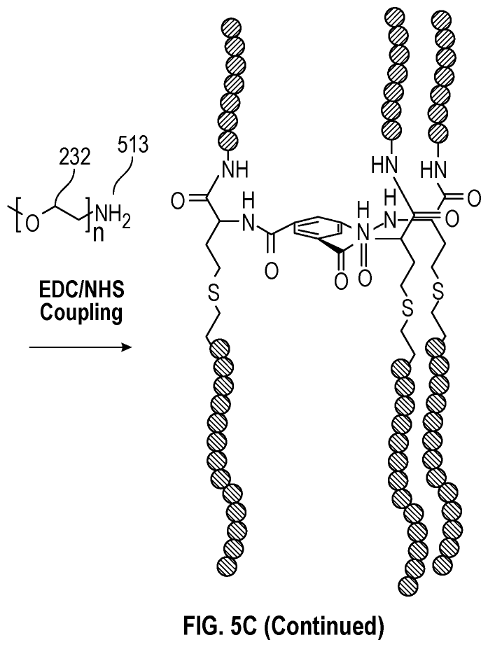

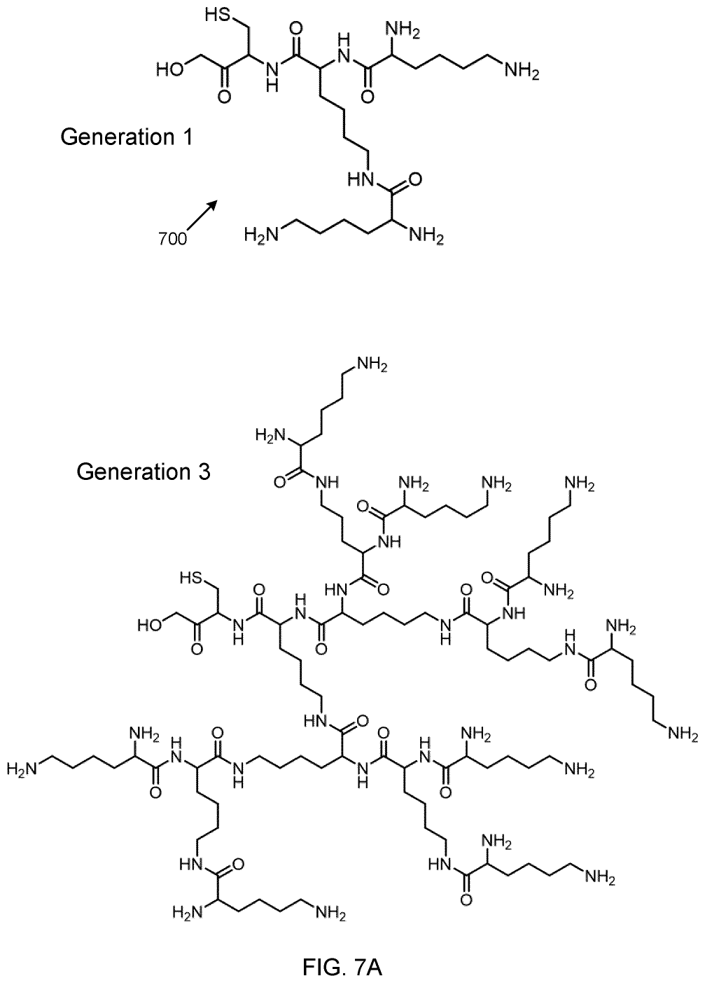

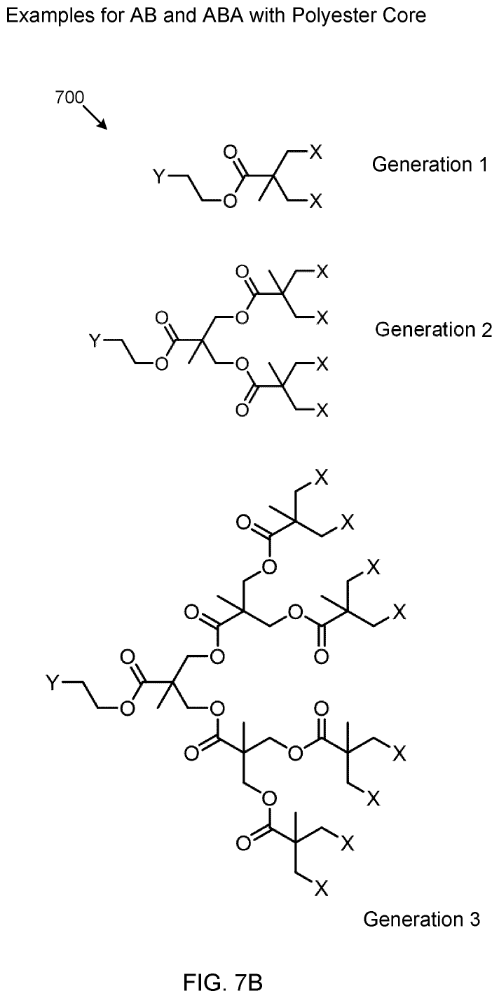

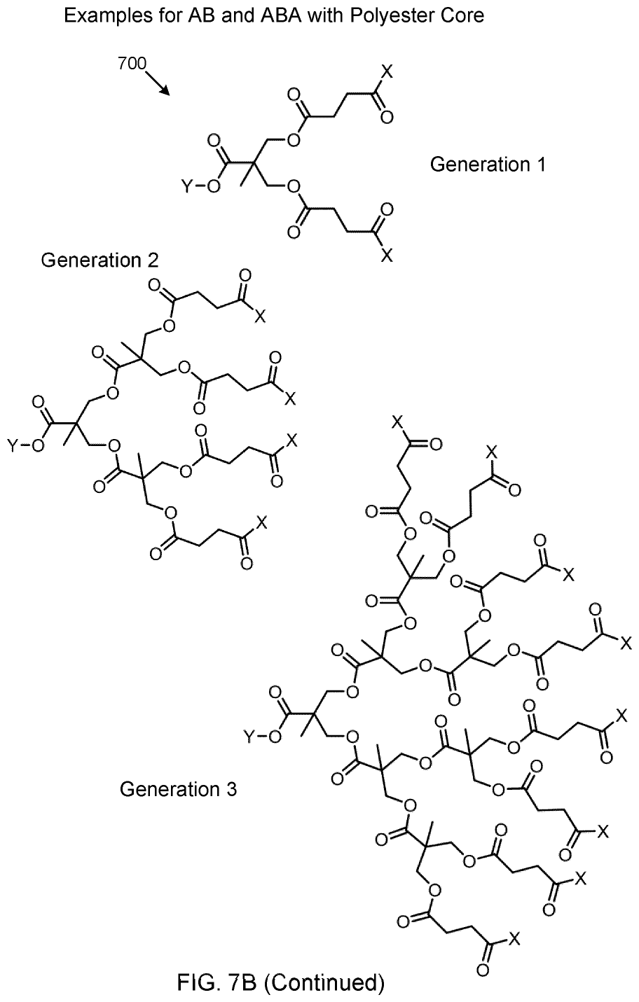

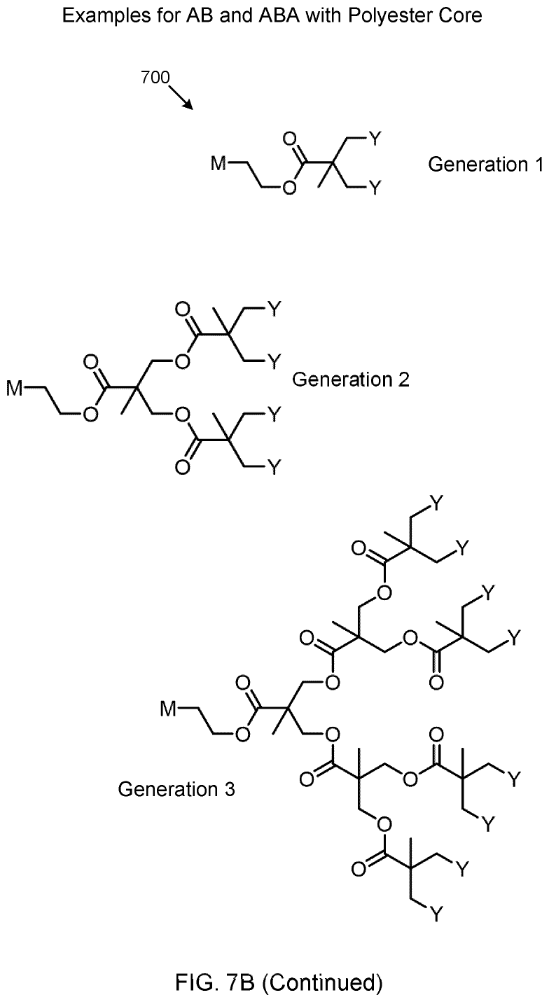

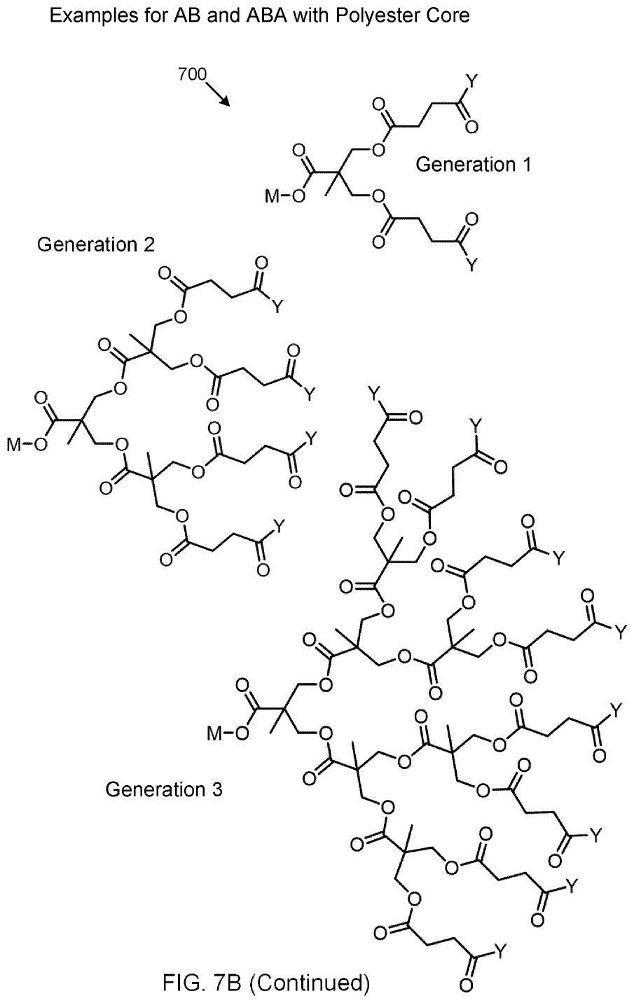

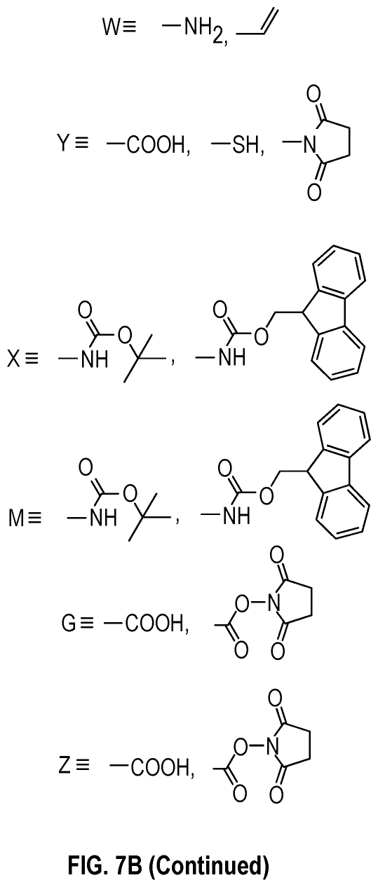

Additionally, the hydrophobic A blocks and the hydrophobic B blocks may be coupled to one another in a similar manner within the amphiphilic units. For example, in the particular example shown in FIG. 2A, within each of the amphiphilic units 221, the hydrophobic blocks 231 may be coupled to respective hydrophilic blocks 232 at a plane. The plane may be substantially parallel to the portion of the layer 201 or 202 in which the particular amphiphilic unit 221 is located. For example, planes P1, P2, and P3 of respective amphiphilic units 221 may be substantially parallel to the portion of layer 201 at the location of those respective amphiphilic units. In the example illustrated in FIG. 2A, the amphiphilic units 221 may include alternating heterograft block copolymers, in which hydrophilic A blocks 232 and hydrophobic B blocks 231 alternate along the length of the amphiphilic unit. Further details regarding alternating heterograft copolymers are provided with reference to FIGS. 4A-4B. In other examples such as described with reference to FIGS. 5A-5C and 7A-7B, the hydrophilic A blocks 232 and hydrophobic B blocks 231 may be coupled to a molecule which acts as a scaffold. Such molecule optionally may lie in the aforementioned plane, e.g., P1, P2, or P3.

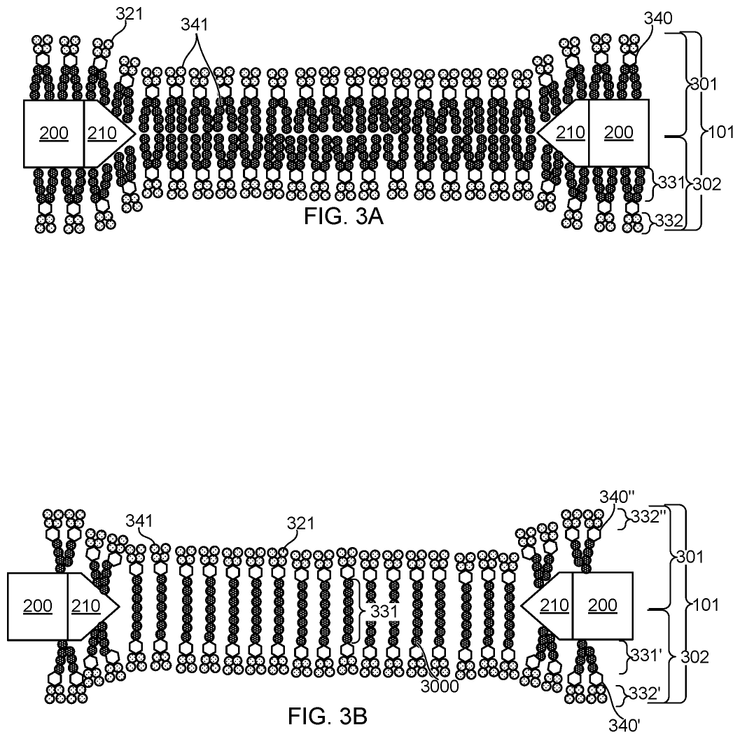

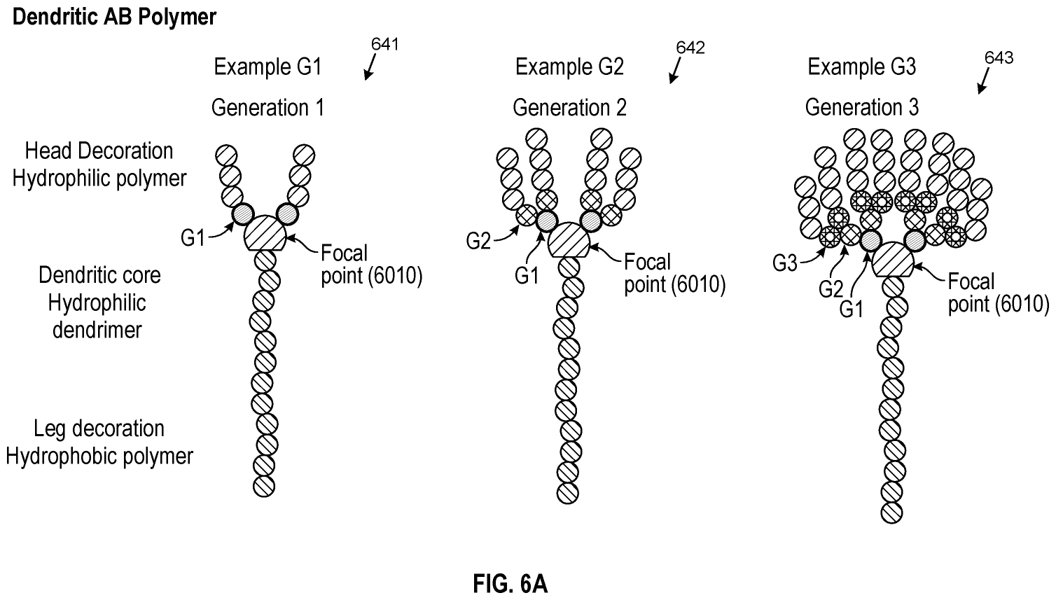

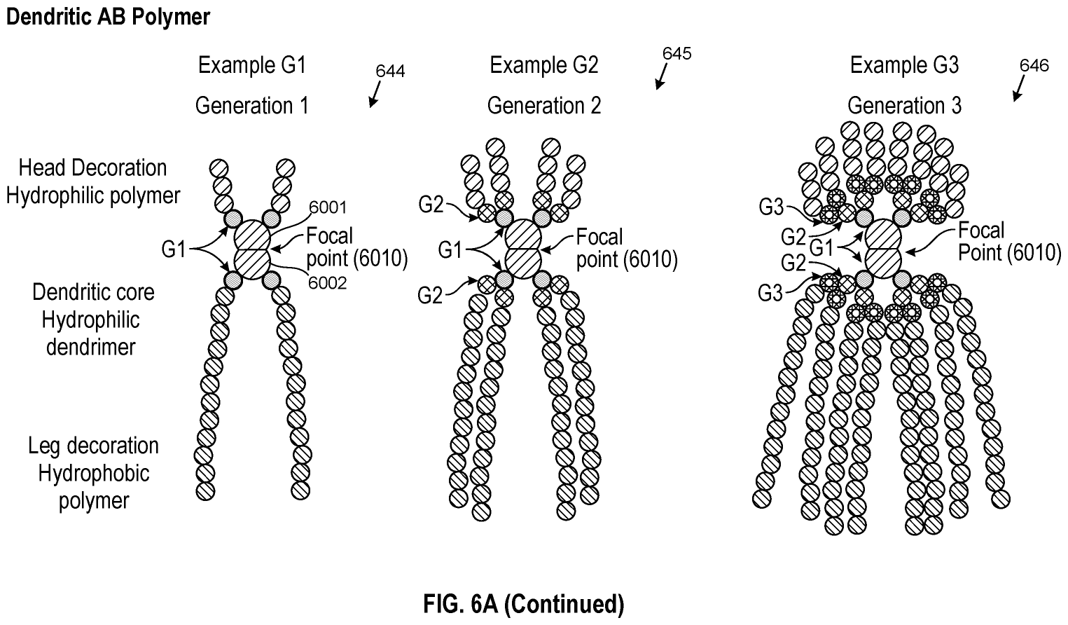

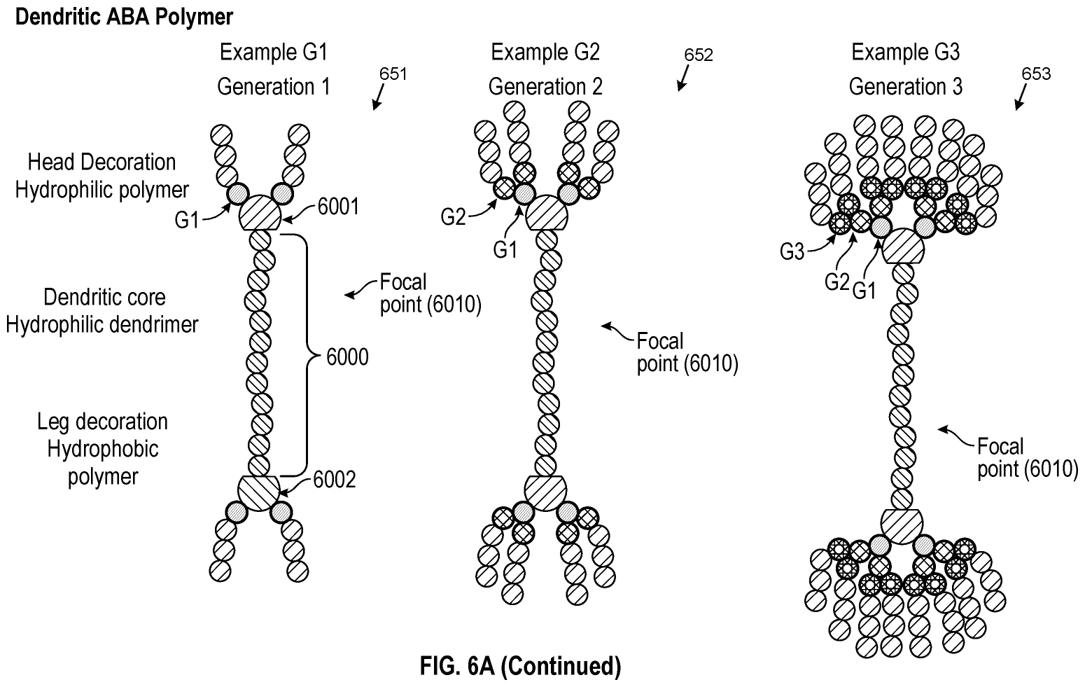

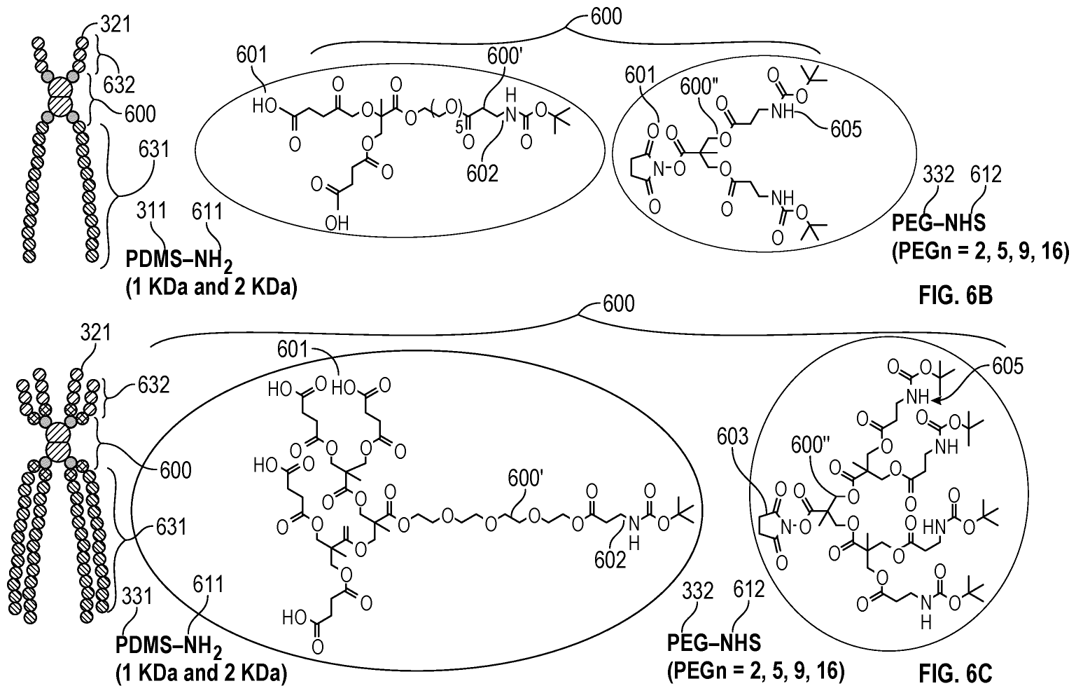

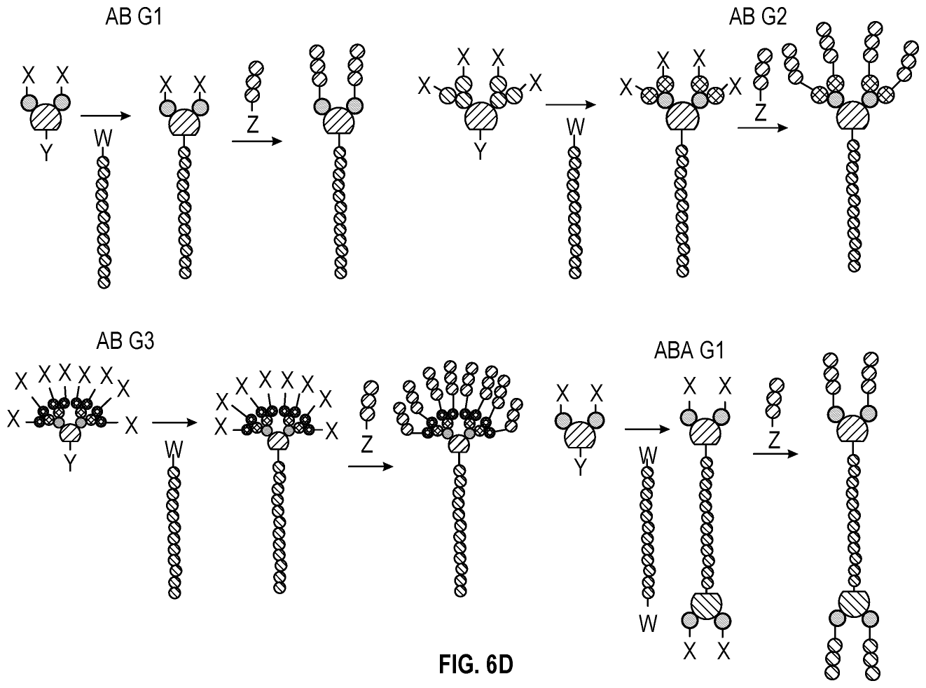

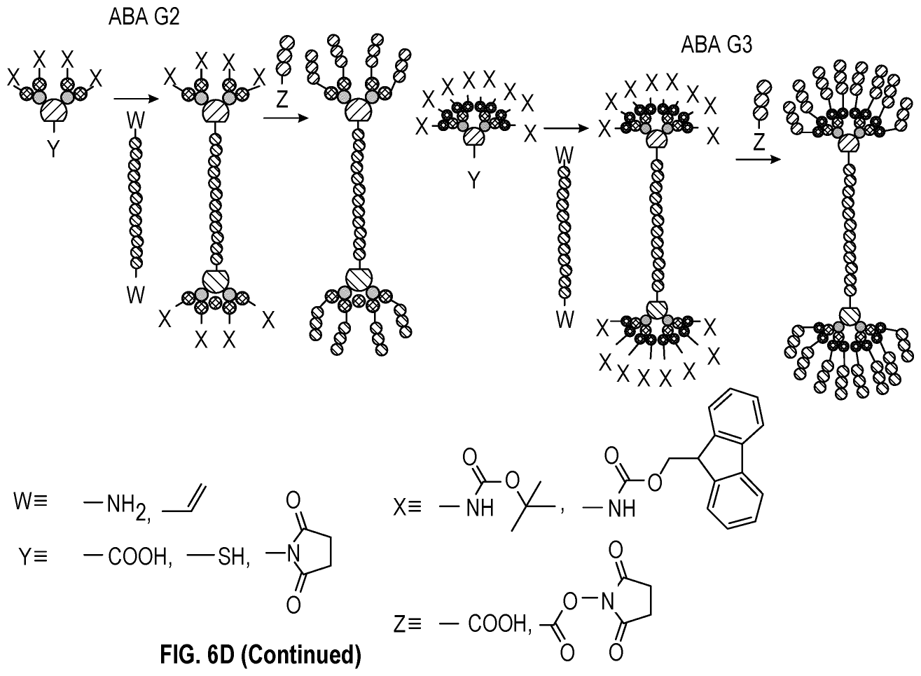

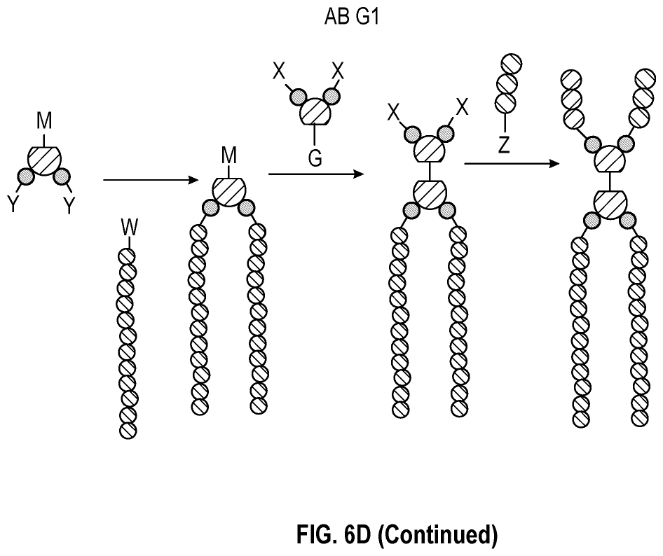

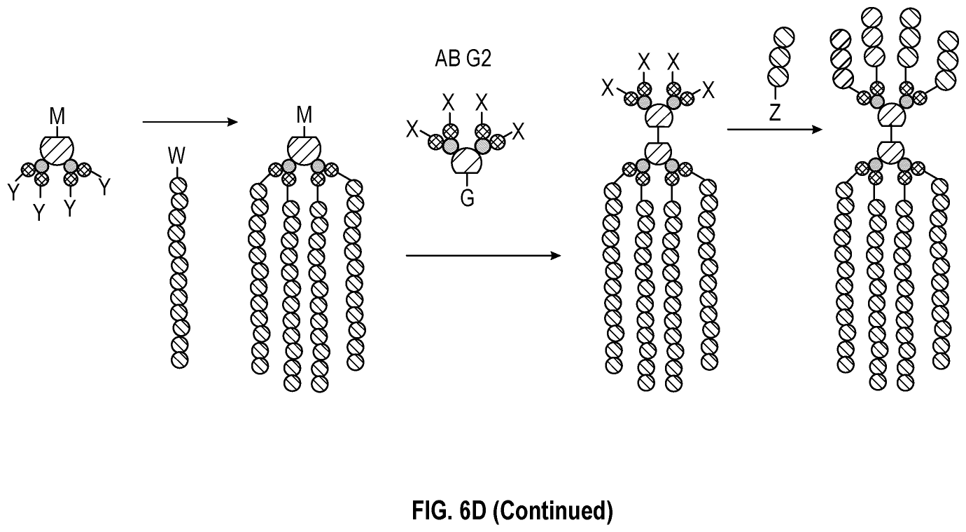

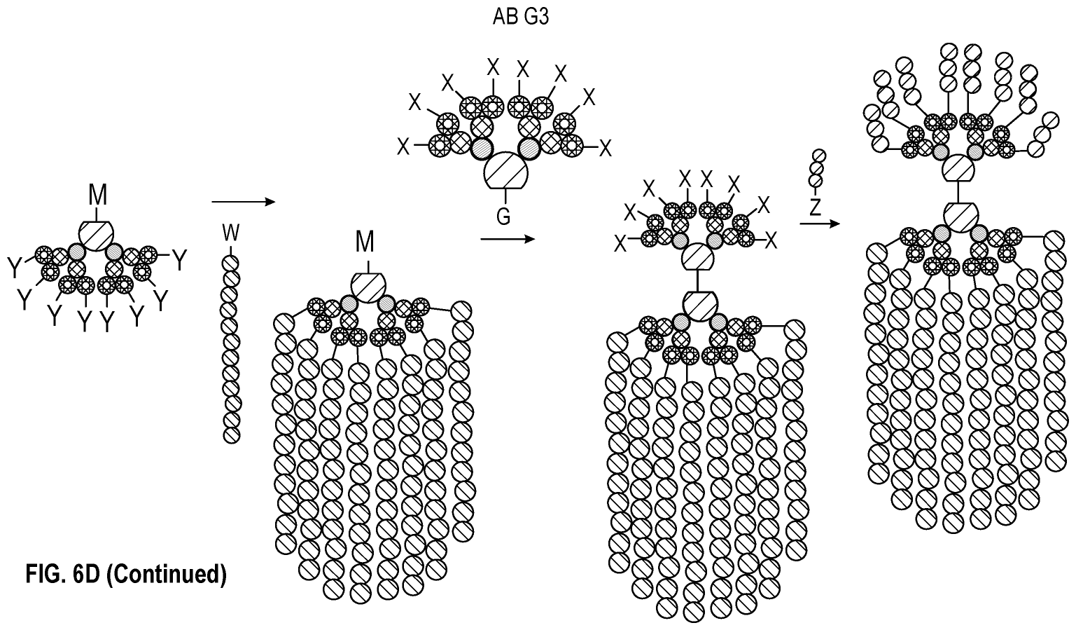

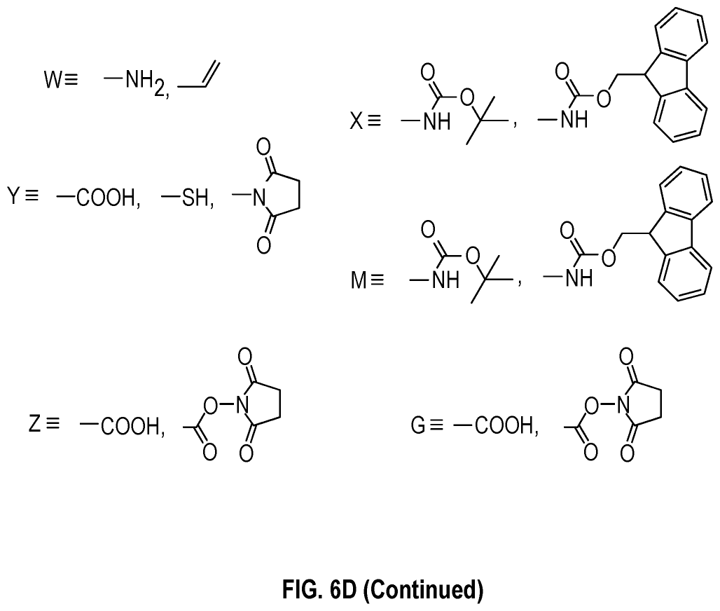

In still other examples, the amphiphilic units may include a dendritic block copolymer. FIGS. 3A-3B schematically illustrate cross-sectional views of other example barriers using amphiphilic units. In the example shown in FIG. 3A, membrane 101 may include first layer 301 including a first plurality of amphiphilic units 321 and second layer 302 including a second plurality of the amphiphilic units and contacting the first plurality of amphiphilic units. Similarly as described with reference to FIG. 2A, the amphiphilic units 321 may include a plurality of hydrophobic “B” blocks 331 (within which circles with darker fill represent hydrophobic monomers) and a plurality of hydrophilic “A” blocks 332 (within which circles with lighter fill represent hydrophilic monomers) coupled directly or indirectly thereto. The amphiphilic units may be substantially the same size as one another, e.g., may have substantially the same molecular mass as one another. For example, the amphiphilic units 321 may include about the same number of hydrophilic A blocks 332 as one another and may include about the same number of hydrophobic B blocks 331 as one another, although the number of hydrophilic A blocks need not be the same as the number of hydrophobic B blocks. Example numbers of blocks are provided elsewhere herein, e.g., with reference to FIGS. 2A-2B and 6A-6D.

The dendritic block copolymer may include dendrons and a core. In the particular example shown in FIG. 3A, within each of the amphiphilic units 321, the dendrons 341 may include hydrophobic blocks 331 and hydrophilic blocks 332. The hydrophobic blocks 331 and hydrophilic blocks 332 may form dendrons that are coupled to one another via core 340. In some examples, such as illustrated in FIG. 3A, the hydrophobic blocks 331 are on different dendrons 341 than the hydrophilic blocks 332. In other examples such as will be described with reference to FIGS. 6A-6D, the hydrophobic blocks 331 may be on at least some of the same dendrons as the hydrophilic blocks 332.

FIG. 3B illustrates an example in which membrane 101 may include substantially a monolayer 301 including a plurality of amphiphilic units 321. Similarly as described with reference to FIG. 2A and FIG. 3A, the amphiphilic units 321 may include a plurality of hydrophobic “B” blocks 331 (within which circles with darker fill represent hydrophobic monomers) and a plurality of hydrophilic “A” blocks 332 (within which circles with lighter fill represent hydrophilic monomers) coupled directly or indirectly thereto. The amphiphilic units may be substantially the same size as one another, e.g., may have substantially the same molecular mass as one another. For example, the amphiphilic units 321 may include about the same number of hydrophilic A blocks 332 as one another and may include about the same number of hydrophobic B blocks 331 as one another, although the number of hydrophilic A blocks need not be the same as the number of hydrophobic B blocks. Example numbers of blocks are provided elsewhere herein, e.g., with reference to FIGS. 2A-2B and 6A-6D.

The dendritic block copolymer may include dendrons and a core. In the particular example shown in FIG. 3B, within each of the amphiphilic units 321, the dendrons 341 may include hydrophobic blocks 331 and first and second sets of hydrophilic blocks 332′, 332″. A first set of the hydrophilic blocks 332′ may form dendrons 341 that are coupled to amphiphilic unit 321 via first core 340′, and a second set of the hydrophilic blocks 332″ may form dendrons 341 that are coupled to amphiphilic unit 321 via a second core 340″. Amphiphilic units 321 shown in FIG. 3B also may include elongated structure 3000 which includes first and second ends and a hydrophobic polymer (that is, a hydrophobic B block 331) disposed between the first and second ends. First core 340′ (which may be referred to as a first dendritic core) may be coupled to the first end of elongated structure 3000, and second core 340″ (first dendritic core) may be coupled to the second end. At regions of the membrane which are disposed on annulus 210 or barrier 200, the amphiphilic units 321 illustrated in FIG. 3B may fold at the hydrophobic B block 331 so that the hydrophilic A blocks 332′, 332″ remain outwardly facing.

In examples such as illustrated in FIGS. 2A-2B and 3A-3B, membrane 101 may be suspended using membrane support 200 defining aperture 230. For example, membrane support 200 may include a substrate having an aperture 230 defined therethrough, e.g., a substantially circular aperture, or an aperture having another shape. Additionally, or alternatively, the barrier support may include one or more features of a well in which the nanopore device is formed, such as a lip or ledge on either side of the well. Nonlimiting examples of materials which may be included in a barrier support are provided further above. An annulus 210 including hydrophobic (non-polar) solvent, and which also may include polymer chains and/or other compound(s), may adhere to membrane support 200 and may support a portion of membrane 101, e.g., may be located within membrane 101 (e.g., between layer 201 and layer 202 illustrated in FIG. 2A, or between layer 301 and layer 302 illustrated in FIG. 3A, or within a bilayer structure formed at the periphery of the membrane by folded molecules such as illustrated in FIG. 3B). Additionally, annulus 210 may taper inwards in a manner such as illustrated in FIG. 2A. An outer portion of the molecules 221 of membrane 101 may be disposed on support 200 (e.g., the portion extending between aperture 230 and membrane periphery 220), while an inner portion of the molecules may form a freestanding portion of membrane 101 (e.g., the portion within aperture 230, a part of which is supported by annulus 210). Although FIGS. 2A-2B illustrate nanopore 110 within barrier 101, it should be understood that the nanopore may be omitted, and that barrier 101 may be used for any suitable purpose. More generally, it should be appreciated that while the barriers described herein are particularly suitable for use with nanopores (e.g., for sequencing, illustratively nanopore sequencing such as described with reference to FIGS. 9-12 and 17), the present barriers need not necessarily have nanopores inserted therein.

Suitable methods of forming membranes which are suspended in a manner such as described with reference to FIGS. 1, 2A-2B, and 3A-3B are known in the art, such as “painting”, e.g., brush painting (manual), mechanical painting (e.g., using stirring bar), and bubble painting (e.g., using flow through the device). Nanopore 110 may be inserted into membrane 101 after the membrane is formed. Nonlimiting examples of techniques for inserting nanopore 110 into the membrane include electroporation, pipette pump cycle, and detergent assisted nanopore insertion. Tools for forming membranes using synthetic polymers and inserting nanopores in the membranes are commercially available, such as the Orbit 16 TC platform available from Nanion Technologies Inc. (California, USA).

It will be appreciated that amphiphilic units 221 and 321 such as respectively described with reference to FIGS. 2A-2B and 3 may be formed using any suitable operation(s) for coupling a predetermined number of hydrophobic blocks to a predetermined number of hydrophilic blocks. In examples such as now will be described with reference to FIGS. 4A-4B and 5A-5C, such coupling may be performed using a predetermined number of addition reactions.

FIGS. 4A-4B schematically illustrate example operations and compositions for use in preparing amphiphilic units, e.g., amphiphilic units 221 described with reference to FIGS. 2A-2B. As illustrated in FIG. 4A, at least some of the hydrophobic blocks 231 may include first moiety and second moieties; and at least some the hydrophilic blocks 232 may include third and fourth moieties. For example, a first hydrophobic block 231 (leftmost element of FIG. 4A, designated 231′) may include first moiety 401 and optionally may not necessarily include any second moieties, e.g., may be monofunctional. Other hydrophobic blocks 231 may include first moiety 401 and additionally may include second moiety 402. Hydrophilic blocks 232 may include third moieties 403 and fourth moieties 404. The addition reactions may include reactions between the first moieties 401 and the fourth moieties 404, and reactions between the second moieties 402 and the third moieties 403. Different elements may be discussed with the use of prime (′) notation to facilitate distinguishing elements from one another.

In a manner such as illustrated in FIG. 4A, the first moieties 401 of hydrophobic blocks 231 and the third moieties 403 of hydrophilic blocks 232 may be protected and then deprotected in a manner as to provide enhanced control of the number of hydrophobic blocks and the number of hydrophilic blocks within amphiphilic units 221. For example, at least some of first moieties 401 may be protected with a first protective group 411 (providing protected first moieties 401′), and at least some of third moieties 403 may be protected with a second protective group 412 (providing protected third moieties 403′); here, use of the prime (′) designation is to facilitate distinguishing the protected moieties from the unprotected moieties. The second protective group 412 may be different from the first protective group 411. As such, different chemistries may be used to selectively remove either the first protective group 411 or the second protective group 412 from the hydrophobic or hydrophilic block which was most recently added, allowing addition of a single block (which includes its own protective group). The protective group of the added block then may be removed, and another single block (which includes its own protective group) may be added. Such a process may be repeated any suitable number of times so as to individually and sequentially couple a desired number of hydrophobic blocks and hydrophilic blocks to one another.

Illustratively, a process flow for forming an amphiphilic unit may include coupling a first moiety 401 of a first hydrophobic block 231′ to a fourth moiety 404 of a first hydrophilic block 232′; here, use of the prime (′) designation is intended to facilitate distinguishing these first blocks from subsequently added blocks. Additionally, first hydrophobic block 231′ may not include a second moiety 402 or protected second moiety 402, and as such may be considered to be monofunctional, whereas other hydrophobic blocks 231 may include both a first moiety 401 and a second protected moiety 402′ and thus may be considered to be bifunctional. The first hydrophilic block 232′ may be coupled to first hydrophobic block 231 via addition reaction between first moiety 401 and fourth moiety 404. As illustrated in FIG. 4A, first hydrophilic block 232′ may include a protected third moiety 403′. The second protective group 412 of protected third moiety 403′ may inhibit any further addition reactions from occurring, and as such only a single hydrophilic block may be coupled to first hydrophobic block 231′ during this operation.

Then, as illustrated in FIG. 4A, after the addition reaction, the second protective group 412 of the added hydrophilic block may be removed to provide third moiety 403 which is available for reaction. The third moiety 403 of that hydrophilic block then may be coupled to the second moiety 402 of another one of the hydrophobic blocks 231 in another addition reaction. As illustrated in FIG. 4A, that hydrophobic block 231 may include protected first moiety 401′. The first protective group 411 of protected first moiety 401′ may inhibit any further addition reactions from occurring, and as such only a single hydrophobic block may be coupled to the previously added hydrophilic block during this operation. Then as illustrated in FIG. 4A, after the addition reaction, the first protective group 411 of the added hydrophobic block may be removed to provide first moiety 401 which is available for reaction. The first moiety 401 of that hydrophobic block then may be coupled to the fourth moiety 404 of another one of the hydrophilic blocks 232 in another addition reaction. In a manner similar to that described with reference to addition of the first hydrophilic block 232′, the added hydrophilic block 232 may include a protected third moiety 403′, the second protective group 412 of which may inhibit any further addition reactions from occurring, and as such only a single hydrophilic block may be coupled to the previously added hydrophobic block 231 during this operation.

As illustrated in FIG. 4A, such operations of deprotecting the most recently added block (whether hydrophobic or hydrophilic), adding a single protected block of the opposite type to that one, deprotecting that block, and then adding another single protected block of the opposite type to that one may be repeated a predetermined number of times so as to generate amphiphilic unit 221 having the desired number of hydrophobic blocks and the desired number of hydrophilic blocks. It will be appreciated that any suitable moieties, any suitable protective groups, and any suitable chemistry for removing such protective groups may be used. For example, as illustrated in FIG. 4A, first moiety 401 may include an amine group (—NH₂), fourth moiety 404 may include a carboxyl group (—COOH), and reaction between the first and fourth moieties may form an amide bond (—NH—CO—) coupling a hydrophobic block to a hydrophilic block being added. Additionally, or alternatively, as illustrated in FIG. 4A, third moiety 403 may include an amine group (—NH₂), second moiety 402 may include a carboxyl group (—COOH), and reaction between the third and second moieties may form an amide bond (—NH—CO—) coupling a hydrophilic block to a hydrophobic block being added. The first moiety 401 (e.g., amine) may be protected using any suitable protective group 411, and likewise the third moiety 403 (e.g., amine) may be protected using any suitable protective group 412. For example, the first and second protective groups 411, 412 may be selected from the group consisting of fluorenylmethoxycarbonyl (Fmoc) and tert-butyloxycarbonyl (Boc). The first and second protective groups may be of different types than one another. Additionally, or alternatively, the first moiety 401 and the third moiety 403 optionally are of the same type as one another (e.g., are both amines). Additionally, or alternatively, the second moiety and the fourth moiety optionally are of the same type as one another (e.g., are both carboxyl, —COOH).

It will also be appreciated that the hydrophobic and hydrophilic blocks may be prepared in any suitable manner and may include any suitable polymer(s). For example, FIG. 4B illustrates a nonlimiting example in which the hydrophilic polymer includes polyethylene oxide (which also may be referred to as PEO or as polyethylene glycol, PEG), the protected third moiety 403′ includes an amine protected using Fmoc 412, and the fourth moiety 404 includes carboxylate. As illustrated in FIG. 4B, the hydrophilic block may be prepared using commercially available components such as lysine having an Fmoc-protected amine group and a terminal amine group available for reaction, and PEG functionalized with n-hydroxy succinimide (NHS). The terminal amine group of the lysine may form an amide linkage with the functionalized PEG, yielding a hydrophilic block 232 including a carboxyl which is ready for reaction with an amine of the most recently added hydrophobic block 231.

FIG. 4B also illustrates a nonlimiting example in which the hydrophobic polymer includes poly(dimethyl siloxane) (PDMS), the protected first moiety 401′ includes an amine protected using Boc 411, and the second moiety 402 includes carboxylate. As illustrated in FIG. 4B, the hydrophobic block may be prepared using commercially available components such as glutamic acid having a Boc-protected amine group, a tert-butyl (tBu)-protected carboxyl, and a terminal carboxyl group available for reaction, and PDMS functionalized with an amine (e.g., terminal amine). The terminal amine group of the PDMS may form an amide linkage with the available terminal carboxyl group of the glutamic acid.

In other examples such as now will be described with reference to FIGS. 5A-5C, amphiphilic molecules 221 are formed using addition reactions that couple the predetermined number of hydrophobic blocks to a molecule, and couple the predetermined number of hydrophilic blocks to the molecule. FIGS. 5A-5C schematically illustrate additional example operations and compositions for use in preparing amphiphilic units. The nonlimiting example illustrated in FIG. 5A includes various nonlimiting options for a molecule 500 to which a predetermined number of hydrophobic blocks and a predetermined number of hydrophilic blocks may be coupled. The molecule 500 may include a predetermined number of first moieties 501 and a predetermined number of second moieties 502 that are different from the first moieties. The first moieties 501 may be used to couple molecule 500 to hydrophobic blocks, and the second moieties 502 may be used to couple the molecule to hydrophilic blocks. For example, the hydrophobic blocks 231 may include respective third moieties 511 that react with the first moieties 501 to couple the predetermined number of hydrophobic blocks to the molecule 500. Similarly, the hydrophilic blocks 232 may include respective fourth moieties 512 that react with the second moieties 502 to couple the predetermined number of hydrophilic blocks to the molecule 500. Third moieties 511 and fourth moieties 512 may be different than one another, such that first moieties 501 selectively react with third moieties 511 and substantially do not react with fourth moieties 512, and second moieties 502 selective react with fourth moieties 512 and substantially do not react with third moieties 511.

Accordingly, it will be appreciated that the number of first moieties 501 in the molecule 500 substantially may correspond to the number of hydrophobic blocks 231 in the amphiphilic molecule 221, and the number of second moieties 502 in the molecule 500 substantially may correspond to the number of hydrophilic blocks 232 in the amphiphilic molecule 221. For example, FIG. 5B illustrates a nonlimiting example of amphiphilic molecule 221 that includes three hydrophobic blocks 231 that are coupled to molecule 500 via respective reaction products R2 between first moieties 501 and third moieties 511. The nonlimiting example of amphiphilic molecule 221 illustrated in FIG. 5B also includes three hydrophilic blocks that are coupled to molecule 500 via respective reaction products R1 between second moieties 502 and fourth moieties 511. The example molecules 500 illustrated in FIG. 5A respectively include one, two, or three first moieties 501 and one, two, or three second moieties 502, but readily may be modified to include any suitable number of first and second moieties 501, 502 so as to be coupled to numbers of hydrophobic and hydrophilic blocks some nonlimiting examples of which are provided elsewhere herein, e.g., with reference to FIGS. 2A-2B. Any suitable molecule 500 may be used as a scaffold to which the hydrophobic blocks 231 and hydrophilic blocks 232 may be coupled. For example, as illustrated in FIGS. 5A-5B, molecule 500 may include a polypeptide which is functionalized so as to include moieties 501 and 502. Custom polypeptides including any suitable type and location of moieties 501, 502 may be ordered from a variety of vendors, such as Genscript Biotech Corporation (Cayman Islands) or Biomatik Corporation (Ontario, Canada). In nonlimiting example, the polypeptide may have a length of between about 2 and about 20 peptides, or a length of between about 4 and about 20 peptides.

FIGS. 5A-5B may be considered to illustrate an example in which the hydrophobic and hydrophilic blocks both are coupled directly to a molecule via respective moieties that are different than one another. However, it will be appreciated that the hydrophobic and/or hydrophilic blocks may be coupled indirectly to the molecule. Indeed, in some examples such as now will be described with reference to FIG. 5C, the hydrophobic blocks respectively may include moieties via which the hydrophilic blocks may be coupled to the molecule, or the hydrophilic blocks respectively may include moieties via which the hydrophobic blocks may be coupled to the molecule.

In the nonlimiting example illustrated in FIG. 5C, molecule 500 may include a functionalized aliphatic molecule or a functionalized aromatic ring (e.g., a functionalized benzene ring, functionalized naphthalene, functionalized anthracene, or functionalized pyrene) including first moieties 501 (e.g., acyl chloride). However, it will be appreciated that any other type of molecule, such as described elsewhere herein, may be used. Hydrophobic blocks 231 may include second moieties 511 (e.g., —NH₂), and may be prepared in any suitable manner. For example, as illustrated in FIG. 5C, a commercially available hydrophobic polymer may include a terminal vinyl group that is reacted with the —SH group of homocysteine in the presence of a photoinitiator and UV light to provide hydrophobic block 231 including second moiety 511. In a manner similar to that described with reference to FIGS. 5A-5B, the second moiety 511 of each of the hydrophobic blocks 231 reacts with a corresponding one of first moieties 501 to couple the hydrophobic blocks to molecule 500. Optionally, the resulting structure may be used to form a barrier in a manner such as described elsewhere herein. That is, one or more hydrophilic block(s) may not necessarily need to be added to the structure in order for such structure to be useful in a barrier. For example, hydrophobic blocks 231 may include third moiety 512 (e.g., carboxyl) which is sufficiently hydrophilic for the structure to be useful in a barrier. Alternatively, third moieties 512 of hydrophobic blocks 231 (which are coupled to molecule 500) may be reacted with fourth moieties 513 (e.g., amine) of hydrophilic blocks 232 so as to couple the hydrophilic blocks directly to the hydrophobic blocks and thus couple the hydrophilic blocks indirectly to molecule 500. In other examples (not specifically illustrated), the hydrophilic blocks may include second moieties that react with the first moieties to couple the predetermined number of hydrophilic blocks to the molecule, and third moieties, and the hydrophobic blocks may include respective fourth moieties that react with the third moieties to couple the predetermined number of hydrophobic blocks to the molecule.

In some examples, the coupling between the hydrophobic blocks 231 and hydrophilic blocks 232 may be performed before the hydrophobic blocks are coupled to molecule 500, for example, using suitable blocks and orthogonal coupling steps. Illustratively an AB block copolymer may be used that includes a suitable reactive moiety (e.g., primary amine) dangling out of the A-B interface (and no other reactive moieties such as amine/hydroxy groups are present elsewhere in the molecule). The blocks could be obtained or generated in a first operation, and in a second operation the copolymers could be coupled to the molecule 500 via the dangling reactive moiety.

Polypeptides and functionalized aromatic rings are nonlimiting examples of molecules to which predetermined numbers of hydrophobic and/or hydrophilic blocks may be coupled. In still other examples, such as will be described with reference to FIGS. 7A-7B, the molecule to which the hydrophobic and hydrophilic blocks are coupled may include a dendrimer. Regardless of the particular type of molecule used, any suitable combination of moieties may be used to couple the hydrophobic and/or hydrophilic blocks to the molecule. For example, the first, second, third, and/or fourth moieties may be selected from the group consisting of: a vinyl moiety, a thiol moiety, an azide moiety, a carboxyl moiety, cysteine moiety, a lysine moiety, a succinimide moiety, a DBCO moiety, an acyl chloride moiety, and a propargyl moiety.

It will be appreciated that any suitable moiety may be used to couple elements of any of the present amphiphilic units to one another, and/or may be used as end groups of the hydrophobic and/or hydrophilic blocks of the present amphiphilic units. Illustratively, such moieties may be selected from the group consisting of: a vinyl moiety, a thiol moiety, an azide moiety, a carboxyl moiety, cysteine moiety, a lysine moiety, a succinimide moiety, a DBCO moiety (or other cycloalkene), an acyl chloride moiety, a propargyl moiety, carboxylic acid, a methyl group, a hydroxyl group, a primary amine, a secondary amine, a tertiary amine, a biotin, an allyl group, an acrylate group, a zwitterionic group, a sulfate, a sulfonate, an alkyl group, an aryl group, an orthogonal functionality, a hydrogen, fluorenylmethoxycarbonyl (Fmoc), and tert-butyl carbamate (NHBoc). For example, such moiet(ies) may be selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen. In some examples, such moiet(ies) are selected from the group consisting of: fluorenylmethoxycarbonyl (Fmoc), tert-butyl carbamate (NHBoc), methyl (CH₃), biotin, carboxyl (COOH), propargyl, azide (N₃), amino (NH₂), hydroxyl (OH), thiol (SH), and sulfonate (SO₃⁻).

Still other operations may be used to prepare other types of amphiphilic units 221. For example, FIGS. 6A-6D schematically illustrate additional example operations and compositions for use in preparing amphiphilic units. FIG. 6A illustrates example configurations of dendrimeric amphiphilic units 321 such as described with reference to FIGS. 3A-3B. FIG. 6A illustrates examples of dendritic AB polymers 641, 642, 643, 644, 645, and 646, corresponding to the amphiphilic units 321 described with reference to FIG. 3A; and dendritic ABA polymers 651, 652, 653, corresponding to the amphiphilic units 321 described with reference to FIG. 3B. The example dendritic AB polymers 641, 642, 643, 644, 645, and 646 respectively include a dendritic core (“focal point”) 6010 corresponding to core 340 described with reference to FIG. 3A, such as hydrophilic dendrimer. The example dendritic AB polymers 641, 642, 643 respectively include two hydrophilic blocks and one hydrophobic block coupled to the dendritic core (641); four hydrophilic blocks and one hydrophobic block coupled to the dendritic core (642); and eight hydrophilic blocks and one hydrophobic block coupled to the dendritic core (643). The example dendritic AB polymers 644, 645, 646, similarly include a dendritic core (focal point 6010) corresponding to core 340 described with reference to FIG. 3A. The example dendritic AB polymers 644, 645, 646 respectively include two hydrophilic blocks and two hydrophobic blocks coupled to the dendritic core (644); to four hydrophilic blocks and four hydrophobic blocks coupled to the dendritic core (645); and eight hydrophilic blocks and eight hydrophobic blocks coupled to the dendritic core (646). Dendritic AB polymers 641, 642, 643, 644, 645, 646 of FIG. 6A, may be used to form a barrier which is substantially a bilayer, e.g., in a manner such as described with reference to FIG. 3A.

The example dendritic ABA polymers 651, 652, 653 shown in FIG. 6A respectively include a dendritic core 6010 coupled to two hydrophilic blocks at a first end and two hydrophilic blocks at a second end (651); a dendritic core coupled to four hydrophilic blocks at a first end and four hydrophilic blocks at a second end (652); and a dendritic core coupled to eight hydrophilic blocks at a first end and eight hydrophilic blocks at a second end (653). In the ABA polymer examples 651, 652, 653, the dendritic core 6010 may include an elongated element 6000 (corresponding to elongated element 3000 described with reference to FIG. 3B) which is coupled to, and separates, first and second dendritic cores 6001, 6002 (first and second focal points, corresponding to cores 340′ and 340″ described with reference to FIG. 3B). The dendritic cores 6001, 6002 may be used as attachment regions for first and second groups of hydrophilic blocks, respectively, while elongated element 6000 may include a hydrophobic polymer which may be used as the B block of an ABA copolymer. The combination of the elongated element 6000 and the first and second dendritic cores 6001, 6002 may be referred to as a polymer “bowtie.” In comparison, in the dendritic AB polymers 644, 645, 646, the dendritic core may include first and second focal points 6001, 6002 (attachment regions for hydrophilic and hydrophobic blocks, respectively) which are directly or indirectly coupled together without the use of an elongated element 6000, or even may be integrally formed with one another. The first and second focal points 6001, 6002 may have different types of functional groups than one another, to permit selective coupling of functionalized hydrophobic and hydrophilic blocks thereto. Dendritic ABA polymers 651, 652, 653 of FIG. 6A, may be used to form a barrier which is substantially a monolayer, e.g., in a manner such as described with reference to FIG. 3B.

It will be appreciated that cores may include any suitable number of branches which are configured to couple to any suitable hydrophobic or hydrophilic block such that the dendrons of the amphiphilic unit 321 described with reference to FIGS. 3A and 3B (some nonlimiting examples of which are provide with reference to FIG. 6A), may include about the same number of hydrophobic blocks as hydrophilic blocks, or may include different numbers of hydrophobic and hydrophilic blocks. Nonlimiting examples of the number of hydrophobic blocks and hydrophilic blocks that may be coupled to core 340 are provided elsewhere herein, e.g., with reference to FIGS. 2A-2B, 3A-3B, and 6A. In one nonlimiting example, amphiphilic unit 321 may include from about 1 to about 8 hydrophobic dendrons, e.g., about 2 to 4 hydrophobic dendrons, or about 4 to 8 hydrophobic dendrons. Additionally, or alternatively amphiphilic unit 321 may include from about 1 to about 8 hydrophilic dendrons, e.g., about 2 to 4 hydrophilic dendrons, or about 4 to 8 hydrophilic dendrons. In some examples, the hydrophobic blocks of amphiphilic unit 321 may be on different dendrons than the hydrophilic blocks. In other examples, the hydrophobic blocks may be on at least some of the same dendrons as the hydrophilic blocks. Additionally, each branch of the core may be functionalized to include a moiety to which a hydrophobic block or a hydrophilic block selectively may be coupled. Example moieties for selectively coupling predetermined numbers of hydrophobic and/or hydrophilic blocks to different portions of molecules are provided elsewhere herein, e.g., with reference to FIGS. 4A-4B and 5A-5C.

FIG. 6B illustrates example operations for forming an amphiphilic unit 321 that includes two hydrophilic dendrons 332, 632, two hydrophobic dendrons 331, 631, and core 600. In the nonlimiting example illustrated in FIG. 6B, core 600 may be assembled using commercially available components such as functionalized, branched PEG 600′ including two first moieties 601 (e.g., carboxyl) to which two second moieties 611 (e.g., amine) of hydrophobic blocks 331 respectively may be coupled; and including third moiety 602 (e.g., Boc protected amine which may be deprotected prior to reaction). Core 600 also may be assembled using branched molecule 600″ including fourth moiety 603 (e.g., NHS) which may react with (deprotected) third moiety 602 to form a core including two additional branches including fifth moieties 605 (e.g., Boc protected amine which may be deprotected prior to reaction) to which two sixth moieties 612 (e.g., NHS) of hydrophilic blocks 332 respectively may be coupled. Other nonlimiting options for molecule 600″, and for moieties which may be reacted with one another to form an amphiphilic unit, are described elsewhere herein such as with reference to FIG. 6D. A wide variety of different generations (number of hydrophilic arms), functional groups, and different coupling routes may be used.

FIG. 6C illustrates example operations for forming an amphiphilic unit 321 that includes four hydrophilic dendrons 332, 632, four hydrophobic dendrons 331, 631, and core 600. In the nonlimiting example illustrated in FIG. 6C, core 600 may be assembled using commercially available components such as functionalized, branched PEG 600′ including four first moieties 601 (e.g., carboxyl) to which four second moieties 611 (e.g., amine) of hydrophobic blocks 331 respectively may be coupled; and including third moiety 602 (e.g., Boc protected amine which may be deprotected prior to reaction). Core 600 also may be assembled using branched molecule 600″ including fourth moiety 603 (e.g., NHS) which may react with third moiety 602 to form a core including four additional branches including fifth moieties 605 (e.g., Boc protected amine which may be deprotected prior to reaction) to which four sixth moieties 612 (e.g., NHS) of hydrophilic blocks 332 respectively may be coupled. Other nonlimiting options for molecule 600″, and for moieties which may be reacted with one another to form an amphiphilic unit, are described elsewhere herein such as with reference to FIG. 6D. A wide variety of different generations (number of hydrophilic arms), functional groups, and different coupling routes may be used.

FIG. 6D schematically illustrates example schemes for synthesizing molecules such as described with reference to FIGS. 6A-6C. A nonlimiting example of a scheme for synthesizing an amphiphilic unit 321 including two hydrophilic blocks 331 and two hydrophobic blocks 331, using the molecules shown in FIG. 6B, is shown below:

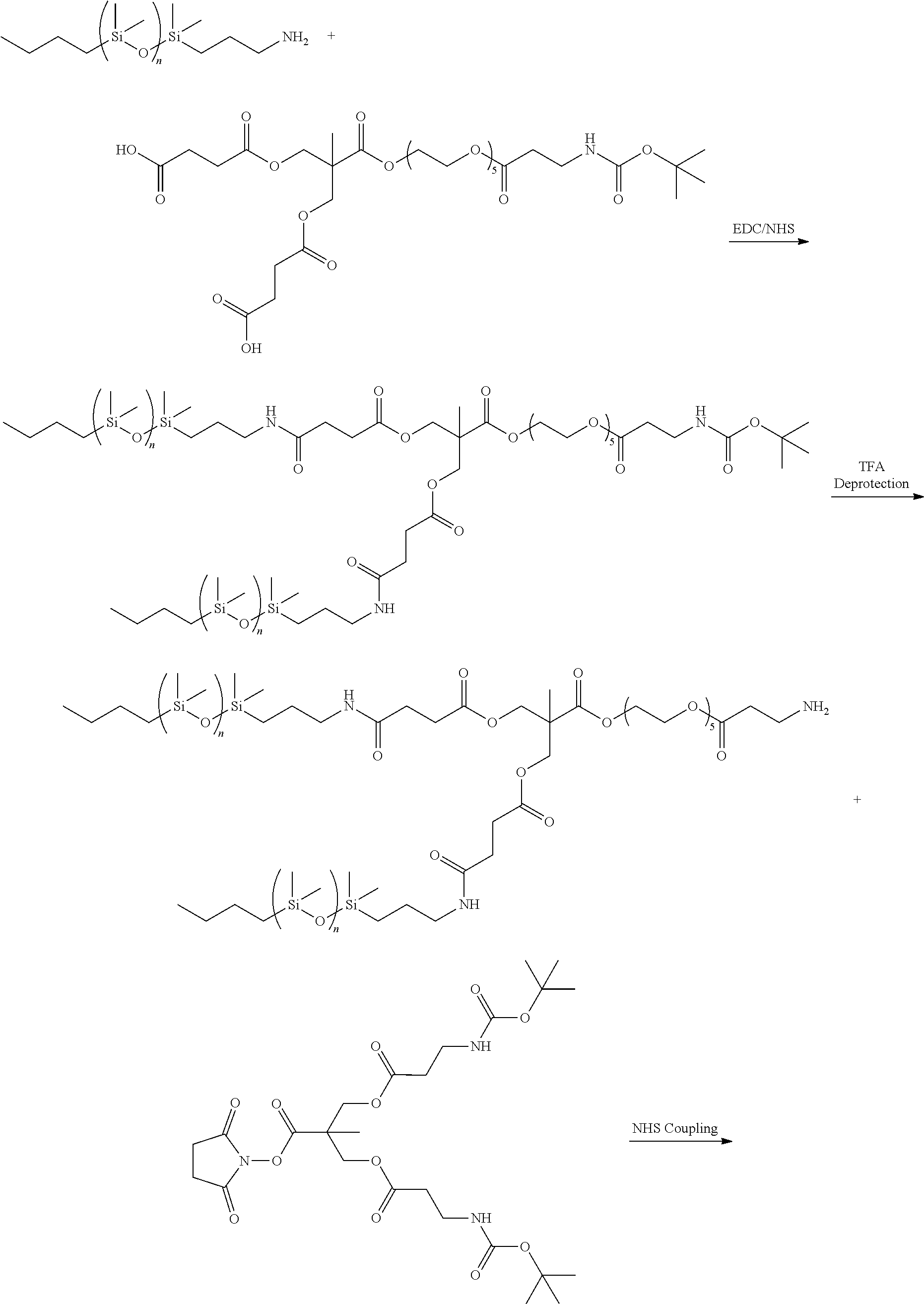

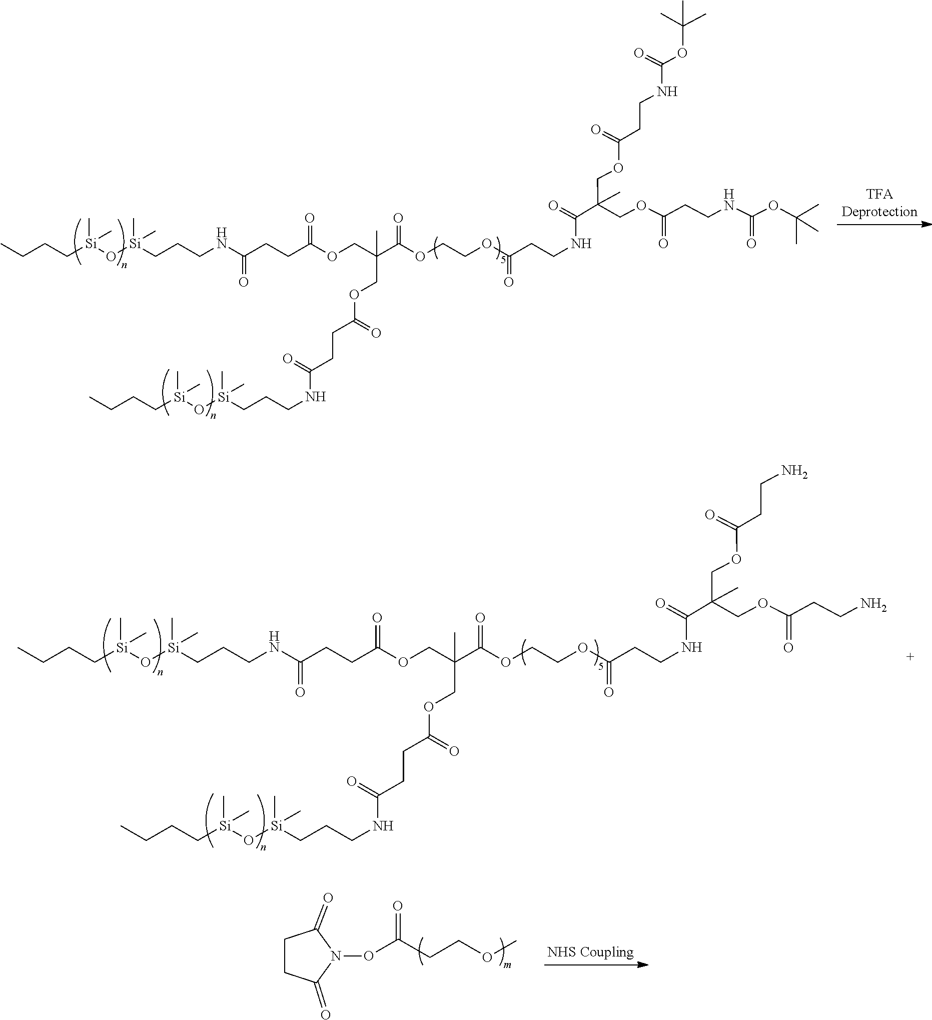

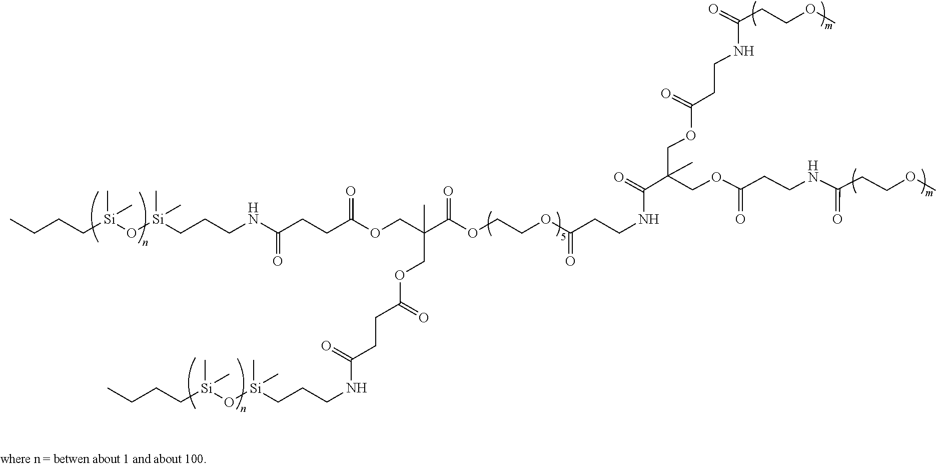

A nonlimiting example of a scheme for synthesizing an elongated a polymer “bowtie” such as described with reference to FIG. 6A, for use in preparing dendritic ABA polymer molecules 651, 652, 653 of FIG. 6A, is below:

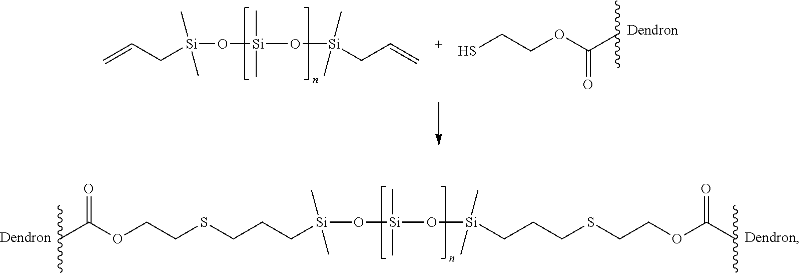

where n=between about 1 and about 100. In the above structure, “Dendron” corresponds to dendritic core 6001 or 6002 described above with reference to FIG. 6A or core 340′ or 340″ described above with reference to FIG. 3B. The elongated structure including PDMS corresponds to elongated structure 6000 described above with reference to FIG. 6A, or elongated structure 3000 described above with reference to FIG. 6A. The structures denoted “Dendron” (corresponding to dendritic core 6001, 6002 or core 340′, 340″) independently may include two or more, four or more, or eight or more functional groups to which hydrophilic blocks respectively may be coupled, corresponding to dendrons 341 and hydrophilic blocks 332′, 332″ described with reference to FIG. 3B.

For example, in the following structure, the R₁ groups may correspond to functional groups that may be used to couple the elongated structure to respective hydrophilic blocks, and the PDMS core may correspond to a hydrophobic block, to form an ABA Generation 1 polymer such as Example G1 shown in example 651 of FIG. 6A.

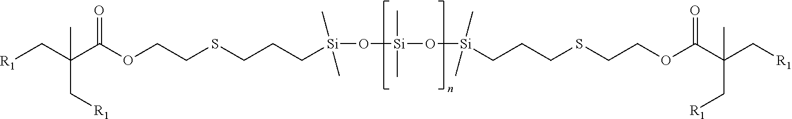

Alternatively, in the above structure, the R₁ groups may be used to couple the elongated structure to additional dendrons that include additional functional groups R₂ such as shown in the below structure, and in which X corresponds to an atom via which the additional dendrons are attached to the elongated structure (e.g., an atom which is in the reaction product between R₁ and the dendron which is added):

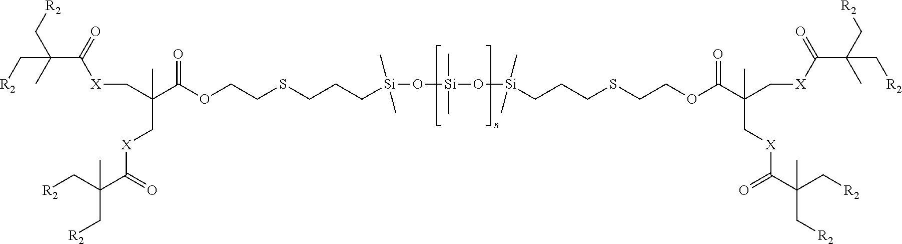

In the structure above, the R₂ groups may be used to couple the elongated structure to respective hydrophilic blocks, and the PDMS core may correspond to a hydrophobic block, to form an ABA Generation 2 polymer such as Example G2 shown in example 652 of FIG. 6A. Alternatively, in the above structure, the R₂ groups may be used to couple the elongated structure to additional dendrons that include additional functional groups (not specifically shown) to which respective hydrophilic blocks may be coupled, to form an ABA Generation 3 polymer such as Example G3 shown in example 653 of FIG. 6A.

Another nonlimiting example of a scheme for synthesizing a dendritic polymer is below:

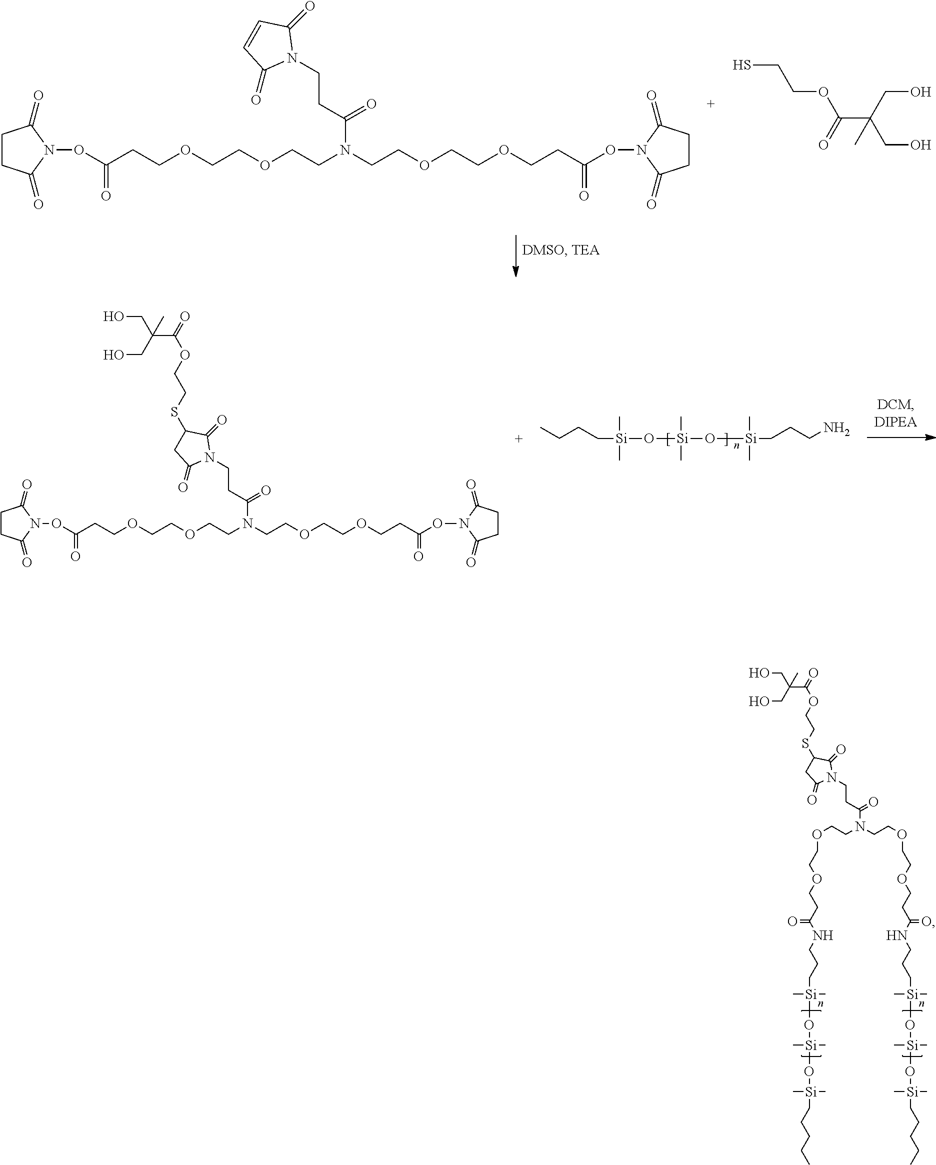

where n=between about 1 and about 100. This example corresponds to amphiphilic units 321 described with reference to FIG. 3A, and to a version of the dendritic AB polymer illustrated in example 644 of FIG. 6A, in which the dendritic AB polymer includes one hydrophilic A block and two hydrophobic B blocks. It will be appreciated that the —OH groups in the hydrophilic block may be replaced with any other suitable R₁ groups such as described in the preceding example. The R₁ groups themselves may be used as the hydrophilic block (e.g., in a generation 1 dendritic AB polymer), or may be coupled to additional moieties, e.g., additional hydrophilic moieties as shown in the example generation 2 dendritic AB polymer shown below:

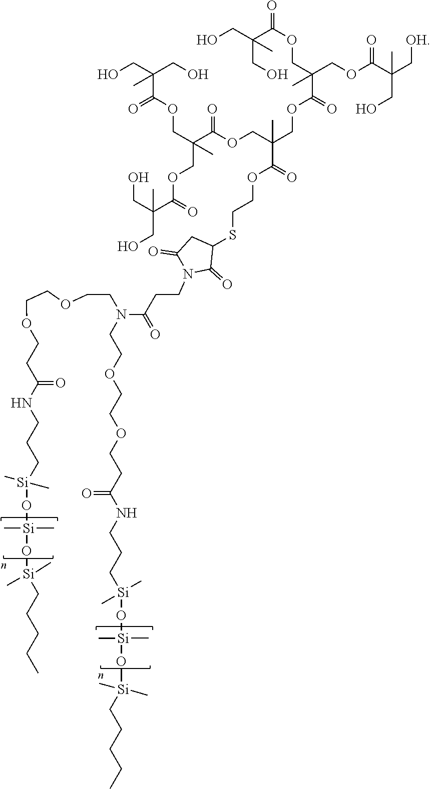

This example corresponds to amphiphilic units 321 described with reference to FIG. 3A, and to a version of the dendritic AB polymer illustrated in example 644 or 645 of FIG. 6A, in which the dendritic AB polymer includes four hydrophilic A blocks and two hydrophobic B blocks. It will be appreciated that the —OH groups in the hydrophilic block may be replaced with any other suitable R₂ groups such as described in the preceding example. The R₂ groups themselves may be used as the hydrophilic block (e.g., in a generation 2 dendritic AB polymer), or may be coupled to additional moieties, e.g., additional hydrophilic moieties as in an example generation 3 dendritic AB polymer described with reference to FIG. 6A.

As noted elsewhere herein, the core of a dendrimer optionally may include a branched polyamide, e.g., polypeptide. For example, FIGS. 7A-7B schematically illustrate additional example operations and compositions for use in preparing amphiphilic units. In the nonlimiting example shown in FIG. 7A, core 700 may include a dendritic polypeptide, which may be used, for example, in core 340, 340′, 340″, focal point 6010, core 6001, and/or core 6002 described elsewhere herein. Nonlimiting examples of the number and type of hydrophobic blocks and hydrophilic blocks that may be coupled to core 700 are provided elsewhere herein, e.g., with reference to FIGS. 2A-2B and 3A-3B. In one nonlimiting example, core 700 includes between about 4 and about 8 branches to which hydrophobic blocks or hydrophilic blocks respectively may be coupled. Branched molecules other than polypeptides alternatively may be used for core 700. In the nonlimiting example illustrated in FIG. 7B, core 700 includes polyester branches to which hydrophobic blocks and hydrophilic blocks may be coupled. Nonlimiting examples of the number and type of hydrophobic blocks and hydrophilic blocks that may be coupled to core 700 are provided elsewhere herein, e.g., with reference to FIGS. 2A-2B and 3A-3B. Nonlimiting examples of reactive moieties that may be used to provide such coupling are illustrated in FIGS. 7A-7B and described elsewhere herein.

Note that in examples such as described with reference to FIGS. 6A-6D and 7A-7B, cores 600 and 700 may be considered to correspond to molecules 500 described with reference to FIGS. 5A-5C, and to include branches to which hydrophobic or hydrophilic blocks respectively may be coupled.

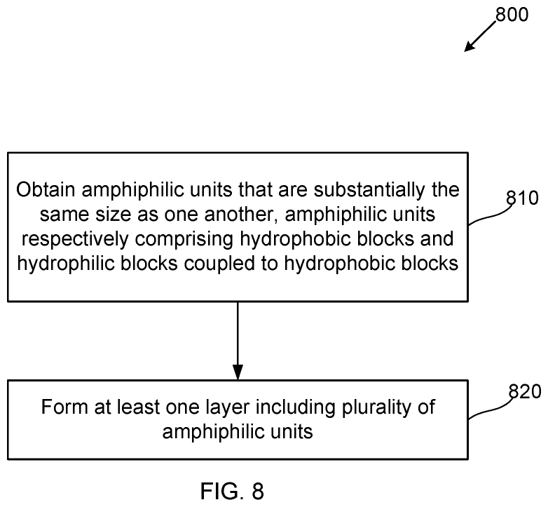

It will be appreciated that any suitable amphiphilic units, such as described herein, may be used to form barriers such as described with reference to FIGS. 1, 2A-2B, and 3A-3B. For example, FIG. 8 illustrates an example flow of operations in a method 800 of forming a barrier using amphiphilic units. Method 800 illustrated in FIG. 8 may include obtaining amphiphilic units that are substantially the same size as one another (operation 810). The amphiphilic units respectively may include hydrophobic blocks and hydrophilic blocks coupled to the hydrophobic blocks. Nonlimiting examples of amphiphilic units, and nonlimiting examples of operations for obtaining amphiphilic units, are described with reference to FIGS. 2A-2B, 3A-3B, 4A-4B, 5A-5C, 6A-6D, and 7A-7B. Example moieties, molecules, and polymers that may be used to obtain the amphiphilic molecules are provided throughout the present disclosure. Method 800 illustrated in FIG. 8 also may include forming at least one layer including a plurality of the amphiphilic units (operation 820). In some examples, operation 820 may include forming first and second layers respectively including first and second pluralities of the amphiphilic units, e.g., such as described with reference to FIGS. 2A-2B and 3A. In other examples, operation 820 may include forming substantially a monolayer including the plurality of the amphiphilic units, e.g., such as described with reference to FIG. 3B. Optionally, nanopore 110 may be inserted into membrane 101 after the membrane is formed. Tools for forming membranes using synthetic polymers and inserting nanopores in the membranes are commercially available, such as the Orbit 16 TC platform available from Nanion Technologies Inc. (California, USA). Optionally, nanopore 110 may be inserted into the resulting membrane. Nonlimiting examples of techniques for inserting nanopore 110 into the membrane include electroporation, pipette pump cycle, and detergent assisted nanopore insertion.

Further details of barriers using polymers which may be included in the nanopore composition and device of FIG. 1 and used in barriers and amphiphilic molecules such as described with reference to FIGS. 2A-2B, 3A-3B, 4A-4B, 5A-5C, 6A-6D, and 7A-7B now will be described. It will be appreciated that such barriers suitably may be adapted for use in any other composition or device, and are not limited to use with nanopores.

Referring again to FIGS. 2A-2B, each of amphiphilic molecules 221 may include any suitable number of hydrophilic blocks 232 (e.g., at least first and second hydrophilic blocks), each being approximately of length “A,” and any suitable number of hydrophobic blocks 231 (e.g., at least first and second hydrophobic blocks) each being approximately of length “B” and coupled to the hydrophilic blocks. The hydrophilic blocks 232 may form the outer surfaces of the barrier 101, e.g., respectively contacting fluid 120 or fluid 120′. The hydrophobic blocks 231 are within the barrier in a manner such as illustrated in FIGS. 2A-2B. Accordingly, layers 201 and 202 each may have a thickness of approximately A+B. In some examples, length A is about 1 repeating unit (RU) to about 100 RU, e.g., about 10 RU to about 80 RU, or about 20 RU to about 50 RU, or about 50 RU to about 80 RU, or about 1 RU to about 20 RU. Additionally, or alternatively, in some examples, length B is about 1 RU to about 100 RU, or about 5 RU to about 100 RU, e.g., about 10 RU to about 80 RU, or about 20 RU to about 50 RU, or about 50 RU to about 80 RU.

Referring again to FIG. 3A, each of amphiphilic molecules 321 may include any suitable number of hydrophilic blocks 332 (e.g., at least one hydrophilic block, and optionally at least first and second hydrophilic blocks), each being approximately of length “A,” and any suitable number of hydrophobic blocks 331 (e.g., at least one hydrophobic block, and optionally at least first and second hydrophobic blocks) each being approximately of length “B” and coupled to the hydrophilic blocks via core 340. The hydrophilic blocks 332 may form the outer surfaces of the barrier 101, e.g., respectively contacting fluid 120 or fluid 120′. The hydrophobic blocks 331 are within the barrier in a manner such as illustrated in FIG. 3A. Accordingly, layers 301 and 302 in the example of FIG. 3A each may have a thickness of approximately A+B, plus the thickness of core 340; and the barrier may have a thickness of approximately 2A+2B, plus twice the thickness of core 340. In some examples, length A is about 1 repeating unit (RU) to about 100 RU, e.g., about 10 RU to about 80 RU, or about 20 RU to about 50 RU, or about 50 RU to about 80 RU, or about 1 RU to about 20 RU. Additionally, or alternatively, in some examples, length B is about 1 RU to about 100 RU, or about 5 RU to about 100 RU, e.g., about 10 RU to about 80 RU, or about 20 RU to about 50 RU, or about 50 RU to about 80 RU.

Referring again to FIG. 3B, each of amphiphilic molecules 321 may include any suitable number of hydrophilic blocks 332 (e.g., at least one hydrophilic block, and optionally at least first and second hydrophilic blocks), each being approximately of length “A,” and any suitable number of hydrophobic blocks 331 (e.g., at least one hydrophobic block, and optionally at least first and second hydrophobic blocks) each being approximately of length “B” and coupled to the hydrophilic blocks via core 340′, core 340″, and elongated structure 3000. The hydrophilic blocks 332 may form the outer surfaces of the barrier 101, e.g., respectively contacting fluid 120 or fluid 120′. The hydrophobic blocks 331 are within the barrier in a manner such as illustrated in FIG. 3A. Accordingly, the barrier in FIG. 3B may have a thickness of approximately 2A+B, plus the thickness of core 340′ plus the thickness of core 340″. In some examples, length A is about 1 repeating unit (RU) to about 100 RU, e.g., about 10 RU to about 80 RU, or about 20 RU to about 50 RU, or about 50 RU to about 80 RU, or about 1 RU to about 20 RU. Additionally, or alternatively, in some examples, length B is about 1 RU to about 100 RU, or about 5 RU to about 100 RU, e.g., about 10 RU to about 80 RU, or about 20 RU to about 50 RU, or about 50 RU to about 80 RU.

The present amphiphilic units may include any suitable combination of hydrophobic and hydrophilic blocks. In some examples, the hydrophilic A block may include a polymer selected from the group consisting of: N-vinyl pyrrolidone, polyacrylamide, zwitterionic polymer, hydrophilic polypeptide, nitrogen containing units, and poly(ethylene oxide) (PEO). Illustratively, the polyacrylamide may be selected from the group consisting of: poly(N-isopropyl acrylamide) (PNIPAM), and charged polyacrylamide, and phosphoric acid functionalized polyacrylamide. Nonlimiting examples of zwitterionic monomers that may be polymerized to form zwitterionic polymers include:

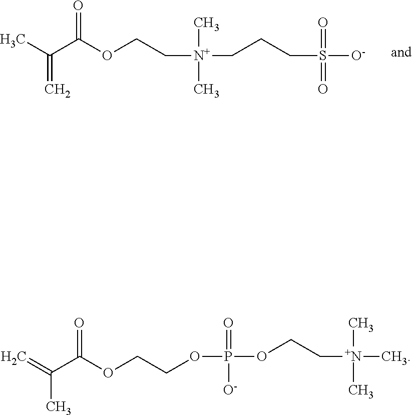 [SMILES: C=C(C)C(=O)OCC[N+](C)(C)CCCS(=O)(=O)[O-] ; C=C(C)C(=O)OCCOP(=O)([O-])OCC[N+](C)(C)C]

Nonlimiting examples of hydrophilic polypeptides include:

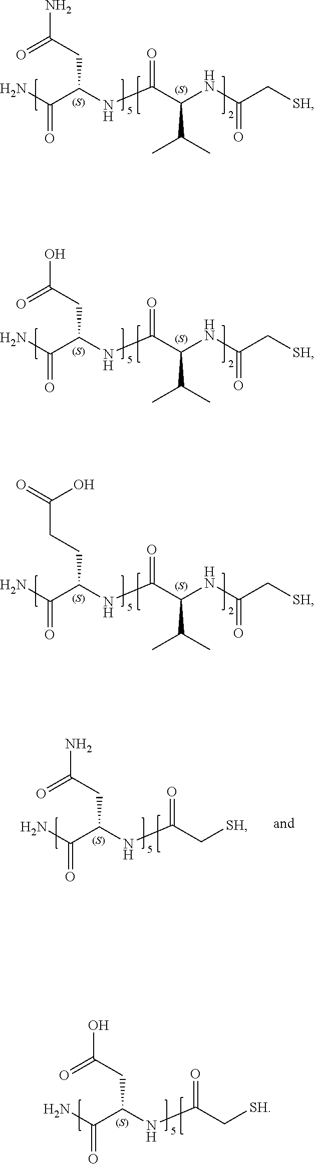

A nonlimiting example of a charged polyacrylamide is

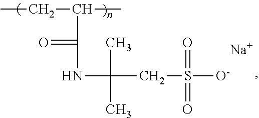

where n is between about 1 and about 100. Nonlimiting examples of nitrogen containing units include:

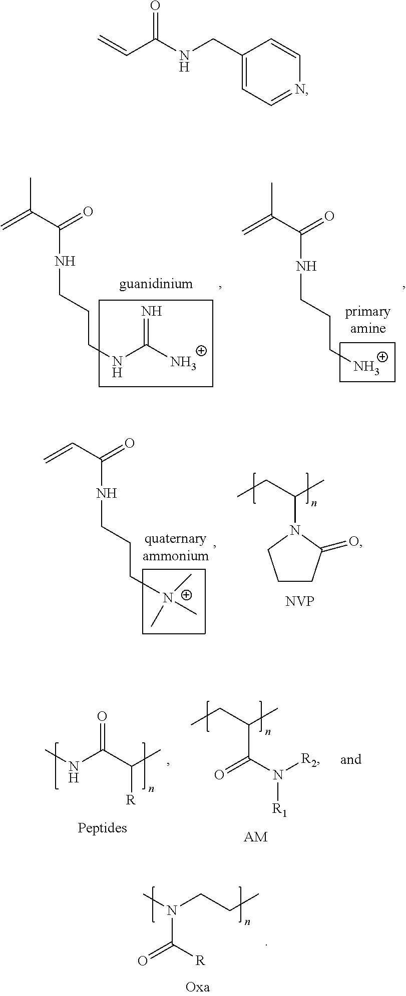

In some examples, the hydrophobic B block may include a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB). Nonlimiting examples of hydrogenated polydienes include saturated polybutadiene (PBu), saturated polyisoprene (PI), saturated poly(myrcene),

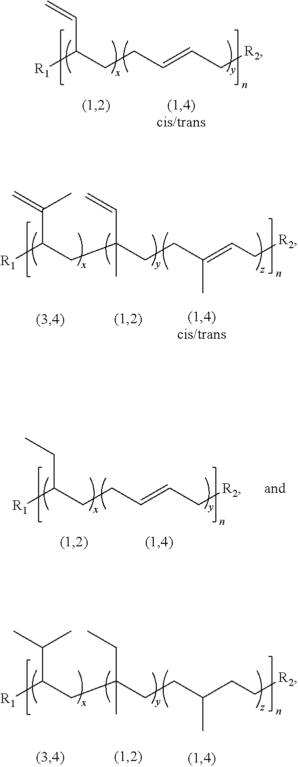

where n is between about 2 and about 100, x is between about 2 and about 100, y is between about 2 and about 100, z is between about 2 and about 100, R₁ is a functional group selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen, and R₂ is a reactive moiety selected from the group consisting of a maleimide group, an allyl group, a propargyl group, a BCN group, a carboxylate group, an amine group, a thiol group, a DBCO group, an azide group, an N-hydroxysuccinimide group, a biotin group, a carboxyl group, an NHS-activated ester, and other activated esters. In other nonlimiting examples of hydrogenated polydienes, R₁ is a reactive moiety selected from the group consisting of a maleimide group, an allyl group, a propargyl group, a BCN group, a carboxylate group, an amine group, a thiol group, a DBCO group, an azide group, an N-hydroxysuccinimide group, a biotin group, a carboxyl group, an NHS-activated ester, and other activated esters. A nonlimiting examples of fluorinated polyethylene is

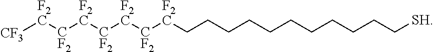 [SMILES: FC(F)(F)C(F)(F)C(F)(F)C(F)(F)C(F)(F)C(F)(F)C(F)(F)C(F)(F)CCCCCCCCCCCS]

Nonlimiting examples of hydrophobic polypeptides include (0<x<1):

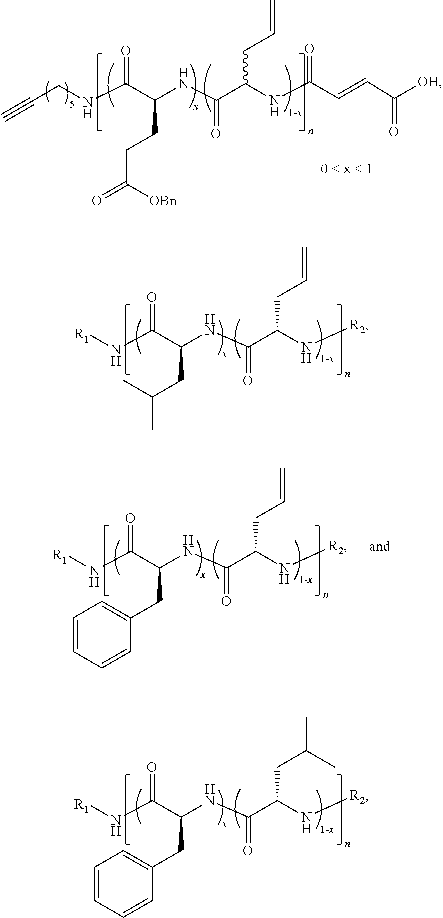

where n is between about 2 and about 100.

In one nonlimiting example, the present amphiphilic molecules include PDMS blocks and PEO blocks. In another nonlimiting example, the present amphiphilic molecules include PBd blocks and PEO blocks. In another nonlimiting example, the present amphiphilic molecules include PIB and PEO blocks. Hydrophobic block(s) may be coupled to hydrophilic block(s) in any suitable manner, e.g., via respective amide bonds or via the products of reactions between moieties. It will be appreciated that any suitable hydrophilic block(s) may be used with any suitable hydrophobic block(s). Additionally, in examples including two or more hydrophilic blocks, those blocks may include but need not necessarily include the same polymers as one another. Similarly, in examples including two or more hydrophobic blocks, those blocks may include but need not necessarily include the same polymers as one another.

The respective molecular weights, glass transition temperatures, and chemical structures of the hydrophobic and hydrophilic blocks, and the numbers of hydrophobic and hydrophilic blocks within the amphiphilic units, suitably may be selected so as to provide the barrier with appropriate stability for use and ability to insert a nanopore. For example, the respective molecular weights of the hydrophobic and hydrophilic blocks may affect how thick each of the blocks (and thus layers of the barrier) are, and may influence stability as well as capacity to insert the nanopore, e.g., through electroporation, pipette pump cycle, or detergent assisted pore insertion. Additionally, or alternatively, the ratio of molecular weights of the hydrophilic and hydrophobic blocks, and the number of hydrophobic and hydrophilic blocks within the amphiphilic units, may affect self-assembly of those amphiphilic units into the layers of the barrier. Additionally, or alternatively, the respective glass transition temperatures (T_{g}) of the hydrophobic and hydrophilic blocks may affect the lateral fluidity of the layers of the barrier; as such, in some examples it may be useful for the hydrophobic and/or hydrophilic blocks to have a T_{g} of less than the operating temperature of the device, e.g., less than room temperature, and in some examples less than about 0° C. Additionally, or alternatively, chemical structures of the hydrophobic and hydrophilic blocks may affect the way the chains get packed into the layers, and stability of those layers.

For nanopore sequencing applications, membrane fluidity can be considered beneficial. Without wishing to be bound by any theory, the fluidity of a block copolymer membrane is believed to be largely imparted by the physical property of the hydrophobic “B” blocks. More specifically, B blocks including “low T_{g}” hydrophobic polymers (e.g., having a T_{g} below around 0° C.) may be used to generate membranes that are more fluid than those with B blocks including “high T_{g}” polymers (e.g., having a T_{g} above room temperature). For example, in certain examples, a hydrophobic B block of the copolymer has a T_{g} of less than about 20° C., less than about 0° C., or less than about −20° C.

Hydrophobic B blocks with a low T_{g} may be used to help maintain membrane flexibility under conditions suitable for performing nanopore sequencing, e.g., in a manner such as described with reference to FIG. 4, 5, 6, 7, or 17. In some examples, hydrophobic B blocks with a sufficiently low T_{g} for use in nanopore sequencing may include, or may consist essentially of, PIB, which may be expected to have a T_{g} in the range of about −75° C. to about −25° C. In other examples, hydrophobic B blocks with a sufficiently low T_{g} for use in nanopore sequencing may include, or may consist essentially of, PDMS, which may be expected to have a T_{g} in the range of about −135° C. (or lower) to about −115° C. In still other examples, hydrophobic B blocks with a sufficiently low T_{g} for use in nanopore sequencing may include, or may consist essentially of, PBd. Different forms of PBd may be used as B blocks in the present barriers. For example, the cis-1,4 form of PBd may be expected to have a T_{g} in the range of about −105° C. to about −85° C. Or, for example, the cis-1,2 form of PBd may be expected to have a T_{g} in the range of about −25° C. to about 0° C. Or, for example, the trans-1,4 form of PBd may be expected to have a T_{g} in the range of about −95° C. to about −5° C. In yet other examples, hydrophobic B blocks with a sufficiently low T_{g} for use in nanopore sequencing may include, or may consist essentially of, polymyrcene (PMyr), which may be expected to have a T_{g} in the range of about −75° C. to about −45° C. In yet other examples, hydrophobic B blocks with a sufficiently low T_{g} for use in nanopore sequencing may include, or may consist essentially of, polyisoprene (PIP). Different forms of PIP may be used as B blocks in the present barriers. For example, the cis-1,4 form of PIP may be expected to have a T_{g} in the range of about −85° C. to about −55° C. Or, for example, the trans-1,4 form of PIP may be expected to have a T_{g} in the range of about −75° C. to about −45° C.

Hydrophobic B blocks with a fully saturated carbon backbone, such as PIB, also may be expected to increase chemical stability of the block copolymer membrane. Additionally, or alternatively, branched structures within the hydrophobic B block, such as with PIB, may be expected to induce chain entanglement, which may be expected to enhance the stability of the block copolymer membrane. This may allow for a smaller hydrophobic block to be used, ameliorating the penalty of hydrophobic mismatch towards an inserted nanopore. Additionally, or alternatively, hydrophobic B blocks with relatively low polarity may be expected to be better electrical insulators, thus improving electrical performance of a device for nanopore sequencing (e.g., such as described with reference to FIG. 9-12 or 17).

### Devices and Methods Using Barriers for Nanopore Sequencing

It will further be appreciated that the present barriers may be used in any suitable device or application. For example, FIG. 9 schematically illustrates a cross-sectional view of an example use of the composition and device of FIG. 1. Device 900 illustrated in FIG. 9 may be configured may include fluidic well 100′, barrier 901 which may have a configuration such as described with reference to FIGS. 1, 2A-2B, 3A-3B, 4A-4B, 5A-5C, 6A-6D, or 7A-7B (and which optionally may be suspended using membrane support 200), first and second fluids 120, 120′, and nanopore 110 in a manner such as described with reference to FIG. 1. In the nonlimiting example illustrated in FIG. 9, second fluid 120′ optionally may include a plurality of each of nucleotides 921, 922, 923, 924, e.g., G, T, A, and C, respectively. Each of the nucleotides 921, 922, 923, 924 in second fluid 120′ optionally may be coupled to a respective label 931, 932, 933, 934 coupled to the nucleotide via an elongated body (elongated body not specifically labeled). Optionally, device 900 further may include polymerase 905. As illustrated in FIG. 9, polymerase 905 may be within the second composition of second fluid 120′. Alternatively, polymerase 905 may be coupled to nanopore 110 or to barrier 901, e.g., via a suitable elongated body (not specifically illustrated). Device 900 optionally further may include first and second polynucleotides 940, 950 in a manner such as illustrated in FIG. 9. Polymerase 905 may be for sequentially adding nucleotides of the plurality to the first polynucleotide 940 using a sequence of the second polynucleotide 950. For example, at the particular time illustrated in FIG. 9, polymerase 905 incorporates nucleotide 922 (T) into first polynucleotide 940, which is hybridized to second polynucleotide 950 to form a duplex. At other times (not specifically illustrated), polymerase 905 sequentially may incorporate other of nucleotides 921, 922, 923, 924 into first polynucleotide 940 using the sequence of second polynucleotide 950.

Circuitry 180 illustrated in FIG. 9 may be configured to detect changes in an electrical characteristic of the aperture responsive to the polymerase sequentially adding nucleotides of the plurality to the first polynucleotide 940 using a sequence of the second polynucleotide 950. In the nonlimiting example illustrated in FIG. 9, nanopore 110 may be coupled to permanent tether 910 which may include head region 911, tail region 912, elongated body 913, reporter region 914 (e.g., an abasic nucleotide), and moiety 915. Head region 911 of tether 910 is coupled to nanopore 910 via any suitable chemical bond, protein-protein interaction, or any other suitable attachment that is normally irreversible. Head region 911 can be attached to any suitable portion of nanopore 910 that places reporter region 914 within aperture 913 and places moiety 915 sufficiently close to polymerase 905 so as to interact with respective labels 931, 932, 933, 934 of nucleotides 921, 922, 923, 924 that are acted upon by polymerase 905. Moiety 915 respectively may interact with labels 931, 932, 933, 934 in such a manner as to move reporter region 914 within aperture 913 and thus alter the rate at which salt 160 moves through aperture 113, and thus may detectably alter the electrical conductivity of aperture 113 in such a manner as to be detected by circuitry 180. For further details regarding use of permanent tethers coupled to nanopores to sequence polynucleotides, see U.S. Pat. No. 9,708,655, the entire contents of which are incorporated by reference herein.

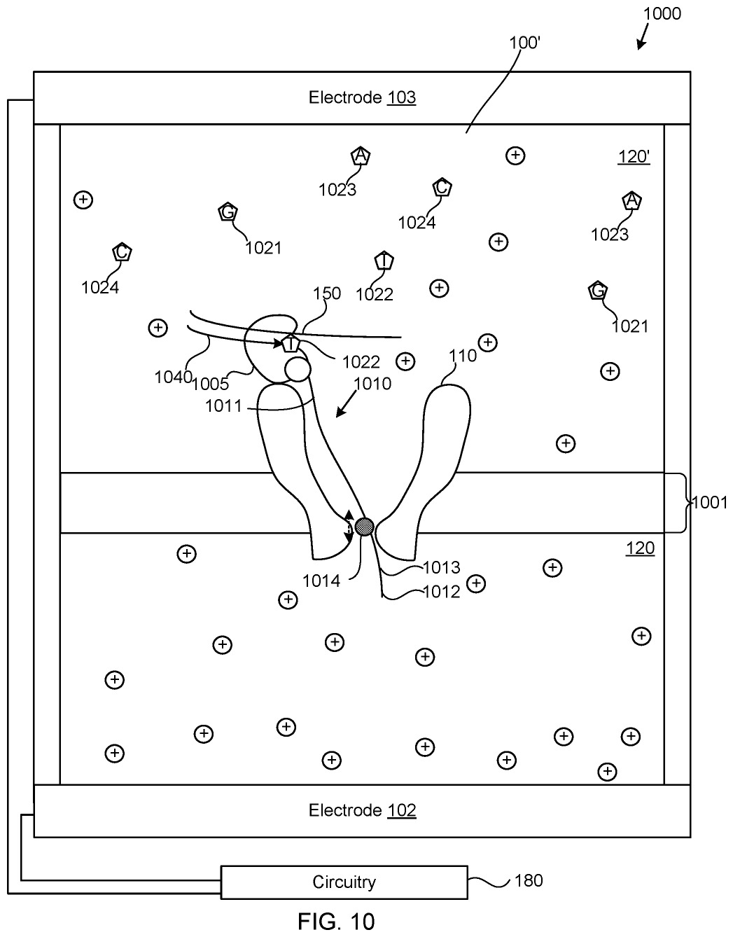

FIG. 10 schematically illustrates a cross-sectional view of another example use of the composition and device of FIG. 1. As illustrated in FIG. 10, device 1000 may include fluidic well 100′, barrier 1001 which may have a configuration such as described with reference to FIGS. 1, 2A-2B, 3A-3B, 4A-4B, 5A-5C, 6A-6D, or 7A-7B (and which optionally may be suspended using membrane support 200), first and second fluids 120, 120′, nanopore 110, and first and second polynucleotides 1040, 1050, all of which may be configured similarly as described with reference to FIG. 9. In the nonlimiting example illustrated in FIG. 10, nucleotides 1021, 1022, 1023, 1024 need not necessarily be coupled to respective labels. Polymerase 1005 may be coupled to nanopore 110 and may be coupled to permanent tether 1010 which may include head region 1011, tail region 1012, elongated body 1013, and reporter region 1014 (e.g., an abasic nucleotide). Head region 1011 of tether 1010 is coupled to polymerase 1005 via any suitable chemical bond, protein-protein interaction, or any other suitable attachment that is normally irreversible. Head region 1011 can be attached to any suitable portion of polymerase 1005 that places reporter region 1014 within aperture 113. As polymerase 1005 interacts with nucleotides 1021, 1022, 1023, 1024, such interactions may cause polymerase 1005 to undergo conformational changes. Such conformational changes may move reporter region 1014 within aperture 113 and thus alter the rate at which salt 160 moves through aperture 113, and thus may detectably alter the electrical conductivity of aperture 113 in such a manner as to be detected by circuitry 180. For further details regarding use of permanent tethers coupled to polymerases to sequence polynucleotides, see U.S. Pat. No. 9,708,655, the entire contents of which are incorporated by reference herein.

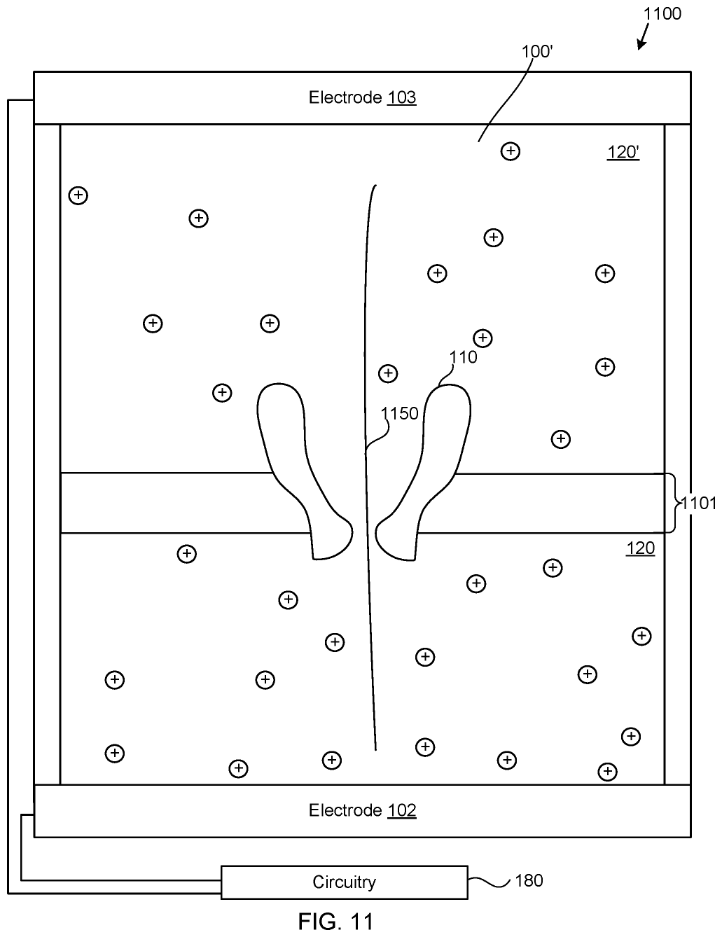

FIG. 11 schematically illustrates a cross-sectional view of another example use of the composition and device of FIG. 1. As illustrated in FIG. 11, device 1100 may include fluidic well 100′, barrier 1101 which may have a configuration such as described with reference to FIGS. 1, 2A-2B, 3A-3B, 4A-4B, 5A-5C, 6A-6D, or 7A-7B (and which optionally may be suspended using membrane support 200), first and second fluids 120, 120′, and nanopore 110, all of which may be configured similarly as described with reference to FIG. 9. In the nonlimiting example illustrated in FIG. 11, polynucleotide 1150 is translocated through nanopore 110 under an applied force, e.g., a bias voltage that circuitry 180 applies between electrode 102 and electrode 103. As bases in polynucleotide 1150 pass through nanopore 110, such bases may alter the rate at which salt 160 moves through aperture 113, and thus may detectably alter the electrical conductivity of aperture 113 in such a manner as to be detected by circuitry 180. For further details regarding use of nanopores to sequence polynucleotides being translocated therethrough, see U.S. Pat. No. 5,795,782, the entire contents of which are incorporated by reference herein.

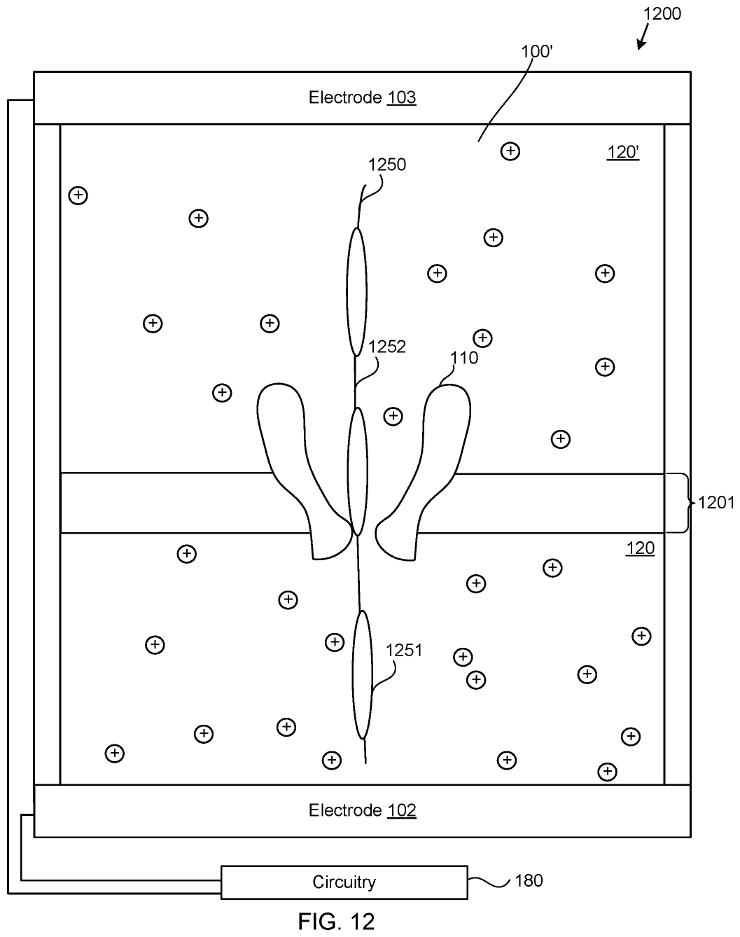

FIG. 12 schematically illustrates a cross-sectional view of another example use of the composition and device of FIG. 1. As illustrated in FIG. 12, device 1200 may include fluidic well 100′, barrier 1201 which may have a configuration such as described with reference to FIGS. 1, 2A-2B, 3A-3B, 4A-4B, 5A-5C, 6A-6D, or 7A-7B (and which optionally may be suspended using membrane support 200), first and second fluids 120, 120′, and nanopore 110 all of which may be configured similarly as described with reference to FIG. 9. In the nonlimiting example illustrated in FIG. 12, surrogate polymer 1250 is translocated through nanopore 110 under an applied force, e.g., a bias voltage that circuitry 180 applies between electrode 102 and electrode 103. As used herein, a “surrogate polymer” is intended to mean an elongated chain of labels having a sequence corresponding to a sequence of nucleotides in a polynucleotide. An XPANDOMER™ is a particular type of surrogate polymer developed by Roche Sequencing, Inc. (Pleasanton, CA). XPANDOMERS™ may be prepared using Sequencing By eXpansion™ (SBX™, Roche Sequencing, Pleasanton CA). In Sequencing by eXpansion™, an engineered polymerase polymerizes xNTPs which include nucleobases coupled to labels via linkers, using the sequence of a target polynucleotide. The polymerized nucleotides are then processed to generate an elongated chain of the labels, separated from one another by linkers which are coupled between the labels, and having a sequence that is complementary to that of the target polynucleotide. For example descriptions of XPANDOMERS™, linkers (tethers), labels, engineered polymerases, and methods for SBX™, see the following patents, the entire contents of each of which are incorporated by reference herein: U.S. Pat. Nos. 7,939,249, 8,324,360, 8,349,565, 8,586,301, 8,592,182, 9,670,526, 9,771,614, 9,920,386, 10,301,345, 10,457,979, 10,676,782, 10,745,685, 10,774,105, and 10,851,405.

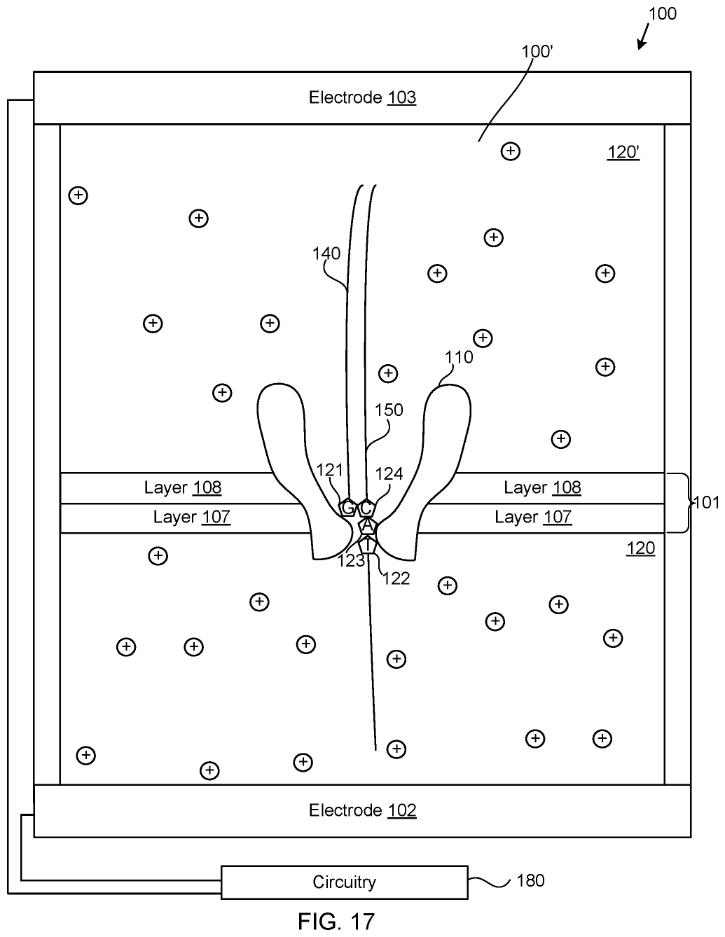

FIG. 17 schematically illustrates a cross-sectional view of another example use of the composition and device of FIG. 1. As illustrated in FIG. 17, device 100 may include fluidic well 100′, barrier 101 which may have a configuration such as described with reference to FIGS. 2A-2C, 14A-14B, 15, and/or 16 (that is, barrier 101 optionally may be suspended using a barrier support, and may include any AB, ABA, or BAB copolymer provided herein), first and second fluids 120, 120′, and nanopore 110 all of which may be configured similarly as described with reference to FIG. 4. In the nonlimiting example illustrated in FIG. 17, a duplex between polynucleotide 140 and polynucleotide 150 is located within nanopore 110 under an applied force, e.g., a bias voltage that circuitry 180 applies between electrode 102 and electrode 103. A combination of bases in the double-stranded portion (here, the base pair GC 121, 124 at the terminal end of the duplex) and bases in the single-stranded portion of polynucleotide 150 (here, bases A and T 123, 122) may alter the rate at which salt 160 moves through aperture 113, and thus may detectably alter the electrical conductivity of aperture 113 in such a manner as to be detected by circuitry 180. For further details regarding use of nanopores to sequence polynucleotides being translocated therethrough, see US Patent Publication No. 2023/0090867 to Mandell et al., the entire contents of which are incorporated by reference herein.

### Working Examples

The following examples are intended to be purely illustrative, and not limiting of the present invention unless otherwise recited in the claims.

### Example 1

The following generation 1 ABA polymer (ABA G1), having two hydrophilic blocks each including a single PEO RU and two hydroxyl groups, and a single hydrophobic block using PDMS in which the average n was approximately 44, was prepared using the synthetic scheme provided further above:

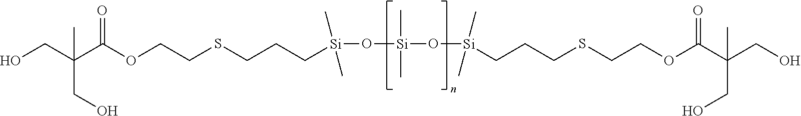

The generation 2 ABA polymer shown below (ABA G2), having a single PEO RU, four hydrophilic groups each with two hydroxyl groups (for a total of eight hydroxyl groups), and a single PDMS hydrophobic block, was generated by using the reactant

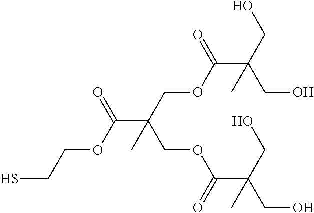 [SMILES: CC(CO)(CO)C(=O)OCC(C)(COC(=O)C(C)(CO)CO)C(=O)OCCS]

to couple additional hydroxylated moieties to the hydroxyl groups of ABA G1:

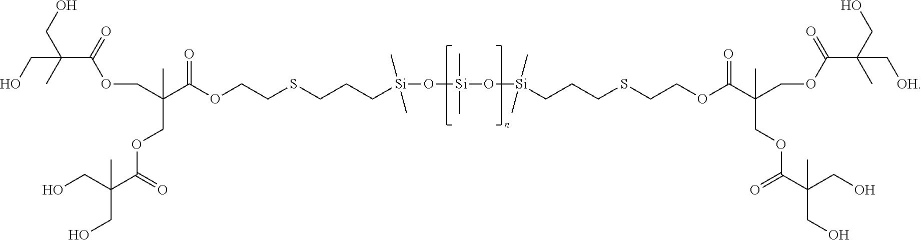

Suspended barriers, such as described with reference to FIG. 3B, were prepared using the ABA G1 and ABA G2 polymers described above. The materials were tested on the Orbit-16 instrument from NanION. This tool allows mechanical painting by rotation of a Teflon stirring bar on top of the chip cavities as well as electrical testing of the membrane/pore construct (membrane capacitance measurement, nanopore I/V curve). The polymers respectively were dissolved in an octane:butanol (95:5 vol) solvent mixture at a concentration of 5 mg/mL prior to testing through suspended membrane formation (also called membrane painting) using a support including a circular aperture such as described with reference to FIGS. 2A-2B, and 3A-3B.

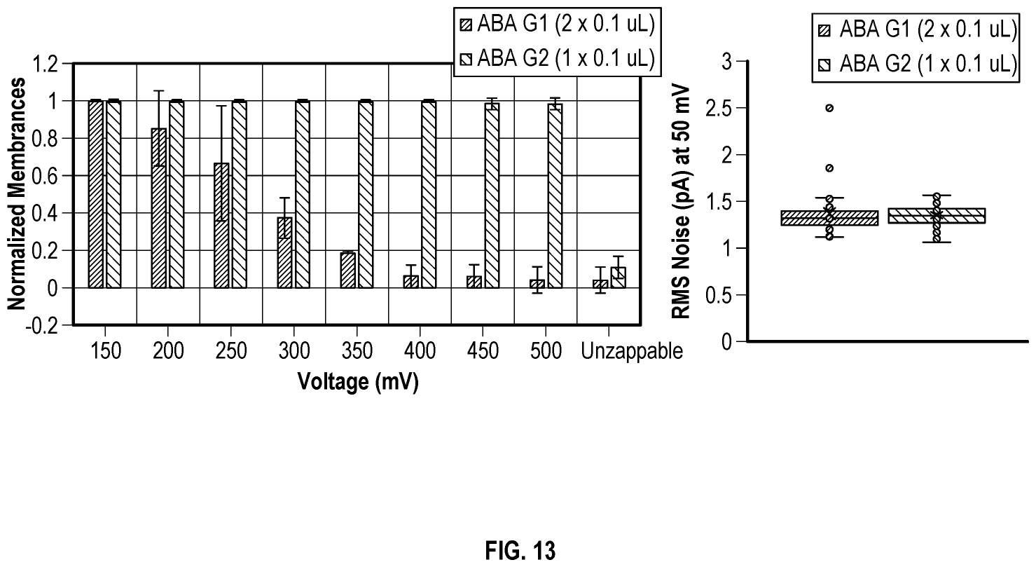

FIG. 13 illustrates plots characterizing properties of barriers made in accordance with examples herein. More specifically, plot (A) in FIG. 13 illustrates a plot describing the breakdown voltage measured for the membranes respectively including ABA G1 and ABA G2. The voltage across the membranes was increased in steps of 50 mV, and the normalized number of surviving membranes was determined at each step. As illustrated in plot (A) of FIG. 13, the normalized number of surviving ABA G1 barriers gradually decreased as a function of voltage. In comparison, the normalized number of surviving ABA G2 barriers did not significantly decrease as a function of voltage, up to a voltage of around 500 mV. Membranes deemed “unzappable” were those which remained stable at a voltage of about 1 V, the highest voltage that could be generated using the system, for at least about 100 ms. Plot (B) in FIG. 13 illustrates the RMS current noise across the ABA G1 and ABA G2 barriers at a voltage of 50 mV. As illustrated in FIG. 13, the RMS noise is comparable between ABA G1 and ABA G2 barriers, in the range of about 1.3-1.4 pA each.

From the foregoing, it may be understood that additional hydrophilic groups may enhance membrane stability.

### Example 2

The following generation 1 AB polymer (AB G1), having one hydrophilic block including a single PEO RU and two hydroxyl groups, and two hydrophobic blocks including PDMS, was prepared using the synthetic scheme provided further above:

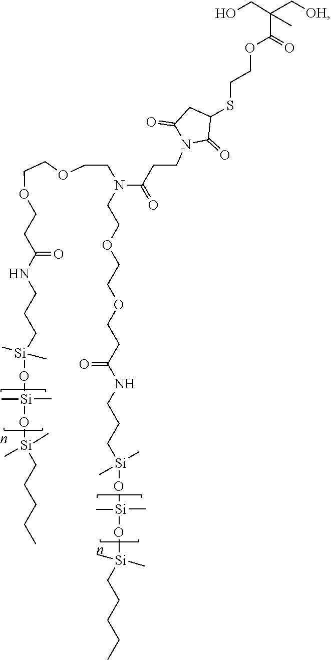

in which the PDMS block had approximately 16 RU.

The following generation 2 AB polymer (AB G2), having a single PEO RU and four hydrophilic groups each with two hydroxyl groups (for a total of eight hydroxyl groups) was generated by coupling additional hydroxylated moieties to the hydroxyl groups of AB G1:

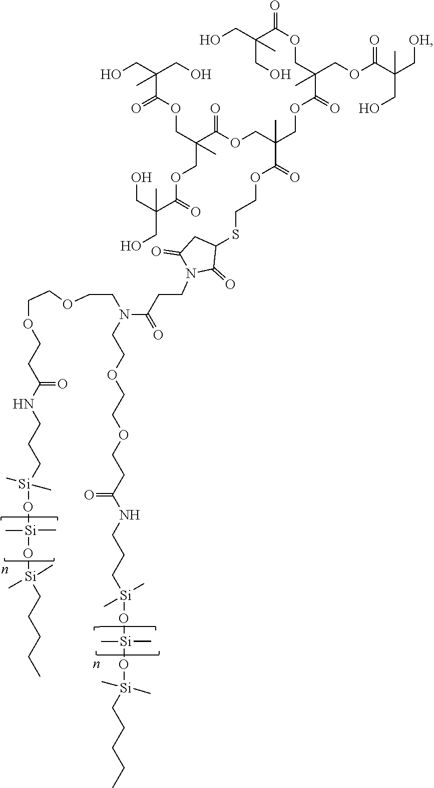

in which the PDMS block similarly had approximately 16 RU.

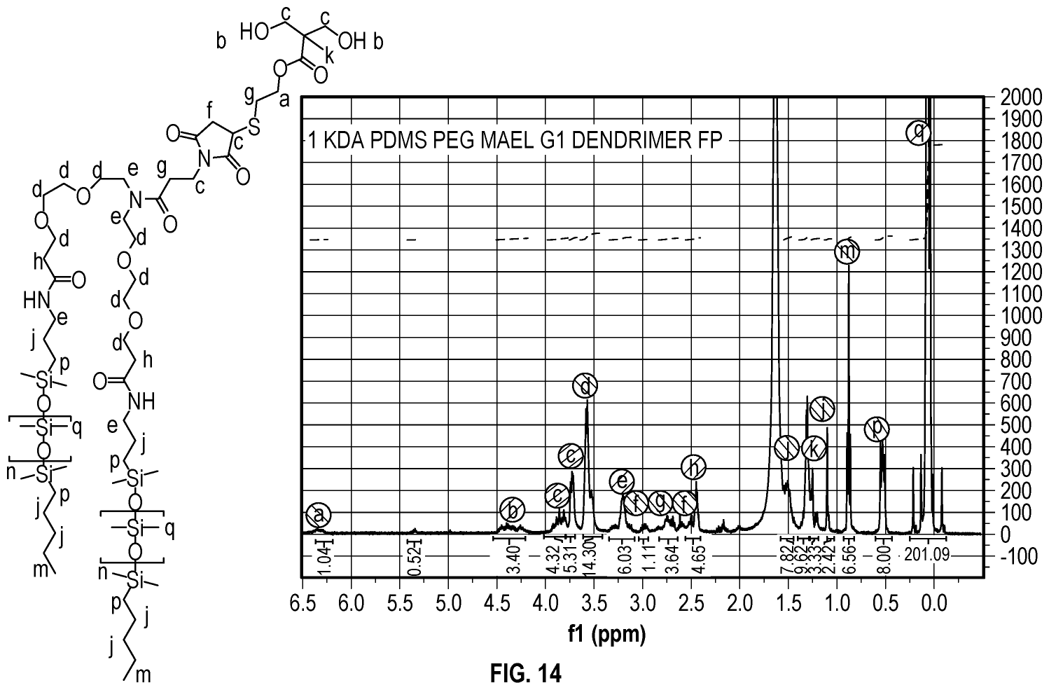

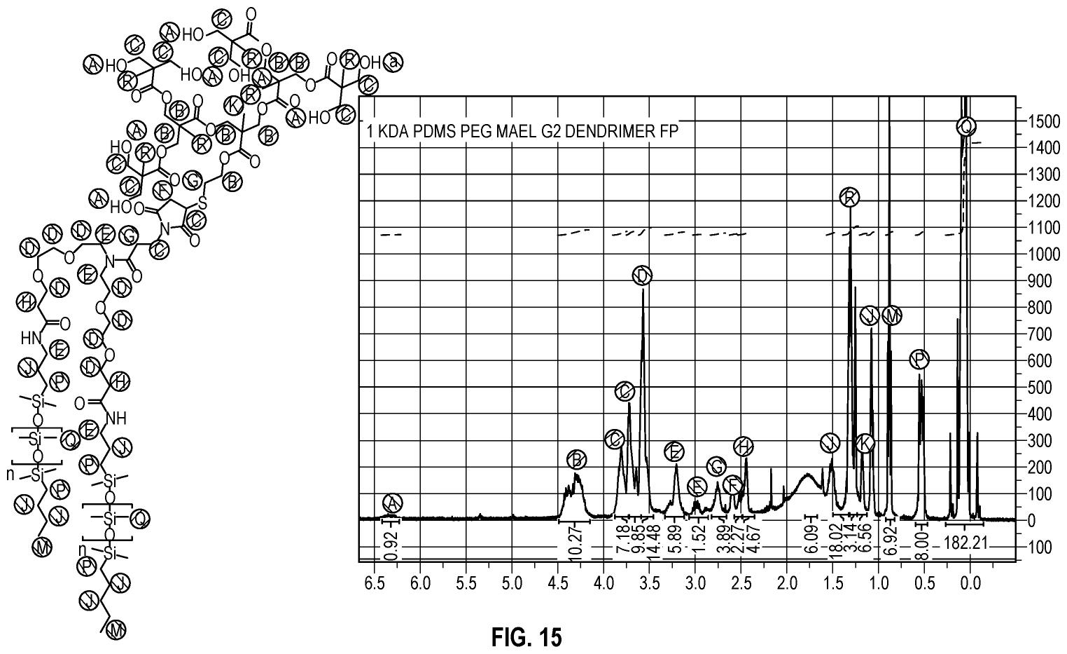

FIGS. 14 and 15 illustrate nuclear magnetic resonance (NMR) spectra of example amphiphilic units. More specifically, FIGS. 14 and 15 respectively illustrate ¹H NMR spectra for the ABA G1 structure and the ABA G2 structure shown above. NMR peaks are labeled to identify the hydrogen atoms responsible for the peak.

Suspended barriers, such as described with reference to FIG. 3A, were prepared using the AB G1 and AB G2 polymers described above. The materials were tested on the Orbit-16 instrument from NanION similarly as in Example 1. The polymers respectively were dissolved in an octane:butanol (95:5 vol) solvent mixture at a concentration of 5 mg/mL prior to testing through suspended membrane formation (also called membrane painting) using a support including a circular aperture such as described with reference to FIGS. 2A-2B, and 3A-3B.

MspA nanopores were inserted into the barriers, and the resulting membrane-pore constructs where characterized. At 50 mV, the current through the membrane-pore construct made using the ABA G1 structure ranged between about 45 pA and about 90 pA, while the current through the membrane-pore construct using ABA G2 structure ranged between about 100 pA and about 110 pA. At the same voltage (50 mV), the RMS current noise for the membrane-pore construct made using the ABA G1 structure ranged between about 3 pA and about 12 pA, while the RMS current noise for the membrane-pore construct using the ABA G2 structure ranged between about 1 pA and about 2 pA.

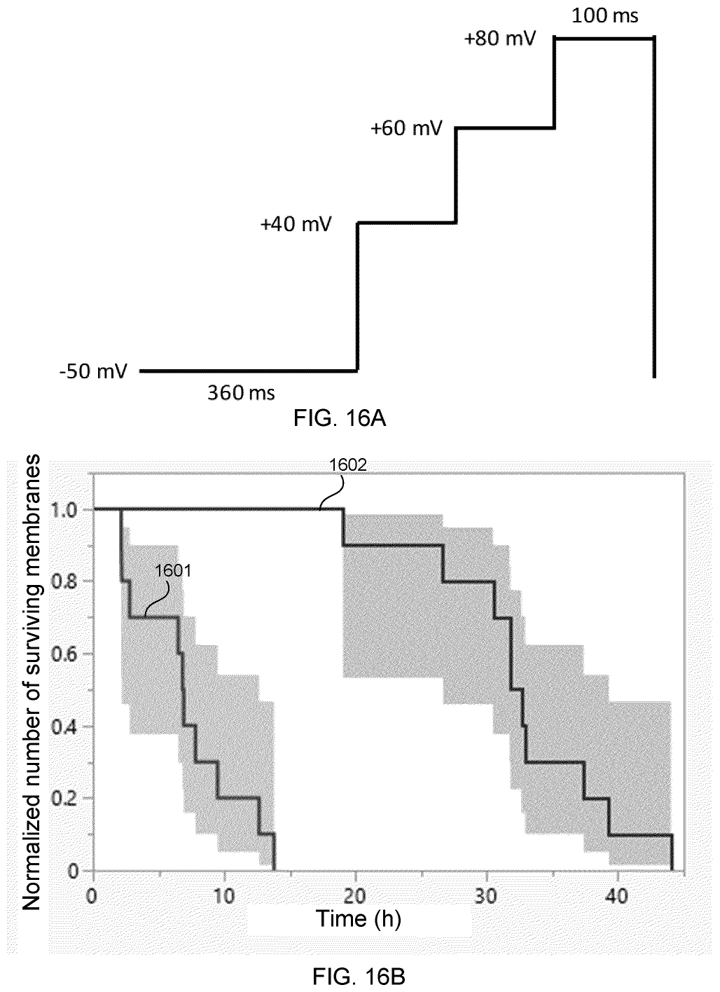

FIG. 16A illustrates a voltage waveform used to assess barrier stability. As shown in FIG. 16A, the voltage across the above-described membrane-pore constructs was held at −50 mV for 360 mS, then was increased to +40 mV for 100 ms, then increased to +60 mV for 100 ms, then increased to +80 mV for 100 ms. Then the voltage was decreased back to −50 mV and the voltage cycle repeated. FIG. 16B illustrates the average survival time of example barriers as a function of time, when subjected the voltage waveform illustrated in FIG. 16A. More specifically, 10 membrane-pore constructs using the ABA G1 structure, and 15 membrane-pore constructs using the ABA G2 structure were subjected to the voltage waveform of FIG. 16A over many hours. As may be seen at plot 1601 in FIG. 16B, the normalized number of surviving membranes for the membrane-pore construct using the ABA G1 structure gradually decreased over the course of about 14 hours, until all of the membrane-pore constructs failed. In comparison, as may be seen in plot 1602 in FIG. 16B, the normalized number of surviving membranes for the membrane-pore construct using the ABA G2 structure remained at 1.0 for about 20 hours; that is, substantially no membrane-pore constructs failed during the first 20 hours of the voltage cycle. After the first 20 hours, the normalized number of surviving membranes for the membrane-pore construct using the ABA G2 structure gradually decreased over the course of about the next 24 hours, until all of the membrane-pore constructs failed around 44 hours into the voltage cycle. Pore ejection was determined to be the most common mode of failure for both the ABA G1 and ABA G2 membrane-pore constructs.

From the foregoing, it may be understood that additional hydrophilic groups may enhance the stability of membranes, particularly membranes into which nanopores are inserted.

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A barrier between first and second fluids, the barrier comprising:
at least one layer comprising a plurality of amphiphilic units,
the amphiphilic units being substantially the same size as one another, and
the amphiphilic units respectively comprising hydrophobic blocks and hydrophilic blocks coupled to the hydrophobic blocks.

2-33. (canceled)

34. A method of forming a barrier between first and second fluids, the method comprising:
obtaining amphiphilic units that are substantially the same size as one another,
the amphiphilic units respectively comprising hydrophobic blocks and hydrophilic blocks coupled to the hydrophobic blocks; and
forming at least one layer comprising a plurality of the amphiphilic units.

35. A method of forming an amphiphilic unit, the method comprising:
coupling a predetermined number of hydrophobic blocks to a predetermined number of hydrophilic blocks using a predetermined number of addition reactions.

36. The method of claim 35, wherein:
at least some of the hydrophobic blocks comprise first and second moieties; and
at least some the hydrophilic blocks comprise third and fourth moieties.

37. The method of claim 36, wherein the addition reactions comprise reactions between the first moieties and the fourth moieties, and reactions between the second moieties and the third moieties.

38. The method of claim 37, further comprising protecting the first moieties with a first protective group; and protecting the third moieties with a second protective group that is different from the first protective group.

39. The method of claim 38, wherein the predetermined number of the addition reactions comprises:
(a) coupling a first moiety of a first hydrophobic block to a fourth moiety of a first hydrophilic block;
(b) removing the second protective group from the third moiety of that hydrophilic block;
(c) coupling the third moiety of that hydrophilic block to the second moiety of another one of the hydrophobic blocks;
(d) removing the first protective group from the first moiety of that hydrophobic block;
(e) coupling the first moiety of that hydrophobic block to the fourth moiety of another one of the hydrophilic blocks; and
(f) repeating operations (b)-(e) a predetermined number of times.

40. (canceled)

41. The method of claim 36, wherein the first moiety and the third moiety are of the same type as one another.

42. The method of claim 41, wherein the first moiety and third moiety are both amines.

43. The method of claim 36, wherein the second moiety and the fourth moiety are of the same type as one another.

44. The method of claim 43, wherein the second moiety and the fourth moiety are both carboxyls.

45. The method of claim 35, wherein the hydrophobic blocks are coupled to the hydrophilic blocks via amide bonds.

46. The method of claim 35, wherein the addition reactions comprise:
coupling the predetermined number of hydrophobic blocks to a molecule; and
coupling the predetermined number of hydrophilic blocks to the molecule.

47. The method of claim 46, wherein:
the molecule comprises a predetermined number of first moieties and a predetermined number of second moieties that are different from the first moieties;
the hydrophobic blocks comprise respective third moieties that react with the first moieties to couple the predetermined number of hydrophobic blocks to the molecule; and
the hydrophilic blocks comprise respective fourth moieties that react with the second moieties to couple the predetermined number of hydrophilic blocks to the molecule.

48. The method of claim 46, wherein:
the molecule comprises a predetermined number of first moieties;
the hydrophilic blocks comprise respective second moieties that react with the first moieties to couple the predetermined number of hydrophilic blocks to the molecule, and third moieties; and
the hydrophobic blocks comprise respective fourth moieties that react with the third moieties to couple the predetermined number of hydrophobic blocks to the molecule.

49. The method of claim 46, wherein:
the molecule comprises a predetermined number of first moieties;
the hydrophobic blocks comprise respective second moieties that react with the first moieties to couple the predetermined number of hydrophobic blocks to the molecule, and third moieties; and
the hydrophilic blocks comprise respective fourth moieties that react with the third moieties to couple the predetermined number of hydrophilic blocks to the molecule.

50. The method of claim 47, wherein the molecule comprises a polypeptide.

51. The method of claim 47, wherein the molecule comprises a functionalized aliphatic molecule or a functionalized aromatic ring.

52. The method of claim 47, wherein the molecule comprises a dendrimer.

53. The method of claim 36, wherein the first, second, third, and fourth moieties are selected from the group consisting of: a vinyl moiety, a thiol moiety, an azide moiety, a carboxyl moiety, cysteine moiety, a lysine moiety, a succinimide moiety, a DBCO moiety, an acyl chloride moiety, and a propargyl moiety.

54-61. (canceled)
